SEQUENCE LISTING (7 sequences)

SEQ ID NO: 1 - protein, 537 aa, Heligmosomoides polygyrus bakeri
  MISC_FEATURE (1)..(537): Glutamate dehydrogenase; UniProtKB Accession Number A0A183FP08
MLSTLARTSGRLIFRRALSSAQMDAHAQVIDDLKPMEEQSNPSFFKMVDYYFDKGATVIEPKLVEEMKSNSMSVMDKKNLVSGILKAIKPVNKVLYITFPIRRDNGEFEVVEAWRAQHSEHRTPTKGGIRYSLDVCEDEVKALSALMTYKCAAVDVPFGGAKGGVKIDPKMYTDYEIEKITRRIAIEFAKKGFLGPGVDVPAPDMGTGEREMGWIADTYAQTIGHLDRDASACITGKPIVAGGIHGRVSATGRGVWKGLEVFAKEPEYMEKIGLTPGLPGKTVIIQGFGNVGLHTMRYLHRAGSKVVGIQEWDCAIHNPAGIHPKELEDWRDQTGSIKNFPGAKNFEPFGDLIYEACDILVPAACEKAIHKENAGRIQAKIIAEAANGPTTPAADKILLERGNCLIIPDMYVNSGGVTVSYFEWLKNLNHVSYGRLSFKYEEDANLMLLQSVQDSLEKAIGKEAPVRPNAQFAAKIAGASEKDIVHSGLEYTMARSGEAIIRTARKYNLGLDMRTAAYANSIEKVYNTYRTAGFTFT

SEQ ID NO: 2 - protein, 152 aa, Heligmosomoides polygyrus bakeri
  MISC_FEATURE (1)..(152): Ferritin; UniProtKB Accession Number A0A183FLG6
LVNIELYASYVYLSMSAYFDRDDVALPNVSKWLRKQSDEEREHAIKLINFQNLRGGRVVFQAVNKPEKDEWGSALEAFQAALALEKFNNQALLDLHAKAAAANDAQMSDFIESTYLGEQVESISEIAKMVTNLKRLGPGMGEYVFDKEQFDS

SEQ ID NO: 3 - protein, 408 aa, Heligmosomoides polygyrus bakeri
  MISC_FEATURE (1)..(408): Aspartate aminotransferase; UniProtKB Accession Number A0A183FI07
MSFFEGIAVAPPIEVFFMNKMYMDEKDPNKVNLTVGAYRTEEGKPWVLPVVREAEKAIAADETLNHEYLPVLGMEAFTEVACKLVLGEQSPAITGGRYFGTQCLSGTGSLRAGAEFLARVLGYKTVLLSNPTWGNHKLVFHNAGFTDVGSYTYWDATNRKVDMQKMLYDLEEAPEKSVIVLHGCAHNPTGMDPTQDQWRQICEVIKRRHLFPFFDIAYQGFASGNPDADAWSIRYFVEQGLEMFIAQSFAKNFGLYNERVGNLCAVVKDPSVLPGYKSQMSLIIRANWSNPPVHGARIVHKVLSTPALRATWDQNIQTMSSRIKQMRSLLQSHLEKLGTPGSWTHITQQIGMFSFTGLTPAQVDHLVKKHKIFLLKNGRISVCGLNTKNVEYVAKAIDDAVRNVPSNL

SEQ ID NO: 4 - protein, 450 aa, Heligmosomoides polygyrus bakeri
  MISC_FEATURE (1)..(450): Tubulin alpha chain; UniProtKB Accession Number A0A183GTY4
MVGYFREVISIHVGQAGVQIGNACWELYCLEHGIQPDGIMPTKNVNESFTTFFSETGNSRFVPRAIMVDLEPTVVDEIRTGTYKQLFHPEQMITGKEDAANNYARGHYTVGKEIIDLVLDRTRRLADNCSGLQGFLIFHSFGGGTGSGFTSLLMERLSVDYGKKSKLEFSIYPAPQVSTAVVEPYNSILTTHTTLEHSDCAFMVDNEAIYDICRRNLDVERPSYTNLNRIISQVVSSITASLRFEGALNVDLTEFQTNLVPYPRIHFPLATYSPLISAEKAYHEALSVGDITNMCFEPCNQMVKCDPRNGKYMAVCLLYRGDVVPKDVNQAISAIKAKRSIQFVDWCPTGFKVGINYQPPTVVPGGDIAKVPRAVCMLSNTTAIAEAWARLDHKFDLMYAKRAFVHWYVGEGMEEGEFSEAREDLAALEKDYEEVGSDSNEEGYEEGEEY

SEQ ID NO: 5 - protein, 123 aa, Heligmosomoides polygyrus bakeri
  MISC_FEATURE (1)..(123): Histone H2B; UniProtKB Accession Number A0A183FWH9
MPPKPSAKGAKKAAKSQKAARTGDKKKRHRRKESYSVYIYRVLKQVHPDTGVSSKAMSIMNSFVNDVFERIAAEASRLAHYNKRSTISSREIQTAVRLILPGELAKHAVSEGTKAVTKYTSSK

SEQ ID NO: 6 - protein, 13 aa, Heligmosomoides polygyrus bakeri
AQHSEHRTPTKGG

SEQ ID NO: 7 - protein, 12 aa, Heligmosomoides polygyrus bakeri
LKPMEEQSNPSF

(19) United States
(12) Patent Application Publication
(10) Pub. No.: US 2021/0170000 A1
(43) Pub. Date: Jun. 10, 2021

(54) Larval preparation of Heligmosomoides polygyrus bakeri as well as methods of making it and uses thereof

(71) Applicant: Helmholtz Zentrum München Deutsches Forschungszentrum für Gesundheit und Umwelt (GmbH), Neuherberg (DE)

(72) Inventors: Carsten Schmidt-Weber, Muenchen (DE); Julia Esser-Von Bieren, Muenchen (DE)

(21) Appl. No.: 17/044,932

(22) PCT Filed: Apr. 5, 2019
(86) PCT No.: PCT/EP2019/058610

§ 371 Date: Oct. 2, 2020

(30) Foreign Application Priority Data
Apr. 5, 2018 (EP) 18000325.3
Apr. 5, 2018 (EP) 18165899.8

(51) Int. Cl. A61K 39/00; A61K 35/62; A61K 38/17; A61K 38/44; A61K 38/45; C07K 1/14; A61P 37/08; A61P 11/00

(52) U.S. Cl. CPC A61K 39/0003; A61K 35/62; A61K 38/1767; C12Y 104/01002; A61P 11/00; A61K 38/45; C12Y 206/01001; C07K 1/145; A61P 37/08; A61K 38/44

(57) ABSTRACT

The present invention relates to cell-free larval preparations of Heligmosomoides polygyrus bakeri (Hpb) helminths, wherein said larval preparation is obtainable from cells of the L3-developmental stage larva of said Hpb helminths, wherein said larval preparation is capable of modulating the innate mammalian immune system as well as methods of making and uses thereof.

This application contains a Sequence Listing in computer readable form, which is incorporated herein by reference.

### TECHNICAL FIELD

The present invention relates to a cell-free larval preparation of Heligmosomoides polygyrus bakeri (Hpb) helminths, wherein said larval preparation is obtainable from cells of the L3-developmental stage larva of said Hpb helminths, wherein said larval preparation is capable of modulating the innate mammalian immune system as well as methods of making it and uses thereof. The present invention further relates to a treatment of steroid resistant chronic airway inflammation with proteins from the nematode parasite Heligmosomoides polygyrus bakeri.

### BACKGROUND OF THE INVENTION

Chronic inflammatory diseases such as asthma and rhinitis, affect more than 200 million people in Europe, causing 20 billion Euro of health care costs. Therapy resistant diseases account for a large part of these costs and they represent a major unmet clinical need (Dominguez-Ortega et al., 2015). Patients suffering from therapy resistant asthma, nasal polyps and intolerance to painkillers (e.g., aspirin) are particularly difficult to treat. This disease, termed aspirin-exacerbated respiratory disease (AERD), affects around 20% of severe asthma patients (Rajan et al., 2015). Lipid mediators derived from arachidonic acid (AA) are key regulators of asthma and nasal polyp pathology (Adamjee et al., 2006, Cahil and Laidlaw 2014, Birrel at al., 2015, Esser-von Bieren et al., 2017). Particularly, the pro-inflammatory leukotrienes (LTs) are strongly implicated in inflammation and airway remodeling, which is a major unmet clinical need (e.g., Henderson et al., 2002, Liu and Yokomizo 2015). However, current treatments against severe asthma and nasal polyps (e.g., in AERD and cystic fibrosis (CF)) show limited efficacy against the LT pathway and/or major side effects as psychotic events and hepatoxicity. Moreover, current drugs targeting single proteins of the AA metabolism fail to broadly modulate the redundant immunological events leading to the airway inflammation.

Glucocorticosteroids are the most commonly used immunomodulatory drugs and topically inhaled corticosteroids (ICS) represent the first line therapy against AERD and most other forms of chronic airway inflammation today. For severe forms of asthma and chronic airway inflammation, glucocorticosteroids are applied orally (i.e., systemically). Glucocorticosteroids (first-line therapy) show limited efficacy against the production of leukotrienes (e.g., Mondino et al. 2004) and fail to suppress the expression of leukotriene pathway proteins in nasal polyps (e.g., Fernandez-Bertolin et al. 2013), which may explain why nasal polyps are often refractory to glucocorticosteroids-treatment (particularly in AERD and CF patients).

The other “immunomodulatory” (AERD specific) approach against LT-driven airway inflammation is Aspirin desensitization, which requires a life-long regular intake of high doses of aspirin (acetylsalicylic acid). However, such aspirin desensitization raises serious concerns about gastro-intestinal adverse side effects and overall safety; high frequency of non-responders or even worsening of symptoms in asthma patients, which has prompted many physicians to refrain from this therapy approach.

Drugs targeting lipid mediator pathways: cysLT1 receptor antagonists: (e.g., Montelukast/Zafirlukast/Pranlukast); 5-lipoxygenase inhibitor (e.g., Zileuton (Zyflo), which has limited use due to its hepatotoxicity (Joshi et al. 2004)); LTC₄ synthase inhibitor(s) (no approved drugs currently available, but substances with activity in vivo (e.g., rodents) and in human cells are under development (Kleinschmidt et al. 2015)) and EP2 agonists (which show bronchoprotective potential in human airways (Saefholm et al., 2015). Furthermore, leukotriene receptor antagonists (LTRAs), e.g., Montelukast (Singulair), target the signaling, but not the production of cysLTs; the redundancy of cysLT receptors (there are at least 3 different receptors that have currently been identified) makes receptor antagonism a very challenging approach (e.g., Kanaoka and Boyce 2014). Moreover, neurological adverse side effects have been reported as LTRAs cross the blood brain barrier. There are even reports that LTRAs loose efficacy only a couple of weeks after the first intake. Zileuton (Zyflo, 5-lipoxygenase inhibitor) efficiently suppresses the production of LTs and shows efficacy in severe asthma, but its use is rather limited due to its hepatotoxic effects (e.g., Zileuton is not approved in Germany) (Joshi et al., 2004).

LTC₄ synthase inhibitors and FLAP inhibitors are currently under development (Kleinschmidt et al., 2015, Bartolozzi et al., 2017, Werz et al., 2017) and are considered as possible candidates for reducing LT production, e.g., in AERD patients. However, these drugs are not designed to broadly reprogram aberrant immune responses in chronic airway inflammation, which exceed the production of LTs (e.g., eosinophil activation, cytokine production, aberrant PGE2 signaling).

Monoclonal antibodies (anti-IL-5, anti IgE): Anti-IL-5 (e.g. mepolizumab) is currently being tested in several clinical studies including AERD-, nasal polyp- and severe asthma patients. Mepolizumab and omalizumab (anti-IgE) have shown efficacy against different types of severe eosinophilic airway inflammation (including nasal polyposis and asthma) (Rivero et al., 2017, Le Pham et al., 2017). Monoclonal antibodies represent the most recent drugs that were introduced into the clinical practice. However, these so-called “biologicals” have major drawbacks such as high costs, high immunogenicity and need for systemic administration.

Allergen specific immunotherapy (AIT): AIT represents the only curative, immunomodulatory treatment option for allergic airway inflammation. However, AIT often shows limited efficacy and new adjuvants are needed to improve the immunomodulatory effects of AIT (e.g., Chesne et al., 2016). Thus, as most AERD, nasal polyp and CF patients are non-allergic or have a large nonallergic inflammatory disease component, AIT does not represent a treatment option for these patients. Furthermore, AIT often fails to control severe allergic airway inflammation possibly due to insufficient immunomodulation (e.g., limited effects on eosinophil activation) (Gunawardana et al. 2017, Virchow et al. 2016) and AIT has significant adverse side effects (Virchow et al. 2016).

For example, WO 2014039223A1 discloses treatments for AERD including: Aspirin desensitization and high-dose aspirin therapy; a P2Y12 inhibitor; Montelukast; a thromboxane receptor antagonist; a 5-lipoxygenase inhibitor; and zileuton. As nasal polyps are frequently refractory to the above-mentioned treatments, many patients (particularly AERD and CF) undergo multiple sinus surgeries, however, with a high level of recurrence of nasal polyps (Mendelsohn et al. 2011).

In light of the above, immunomodulatory proteins of Hpb or other helminths have not been so far investigated regarding their effects on lipid mediator pathways, efficacy in AERD or use as adjuvants in allergen specific immunotherapy. Thus, the problem to be solved by the present invention could inter alia be seen in identifying a superior (compared to current clinical practices) ways (including products, methods and uses) of balancing mediator production and reducing inflammation in severe types of airway diseases (e.g., AERD, nasal polyposis, severe allergic asthma and cystic fibrosis). Another problem to be solved by the present invention could inter alia be seen in improving the efficacy of allergen specific immunotherapy.

The present invention solves said problems, e.g., by providing immunomodulatory proteins and preparations derived from a L3-larvae of Heligmosomoides polygyrus bakeri (Hpb) helminths. The LT-suppressive and overall anti-inflammatory potential of the Hpb proteins and preparations of the present invention is surprising as Hpb nematode larvae are usually assumed to trigger eosinophilia and LT production.

### SUMMARY OF THE INVENTION

The present invention relates to a cell-free larval preparation of Heligmosomoides polygyrus bakeri (Hpb) helminths, wherein said larval preparation is obtainable from cells of the L3-developmental stage larva of said Hpb helminths, wherein said larval preparation is capable of modulating the innate mammalian immune system.

The present application satisfies this demand by provision of the preparations, polypeptides, compositions, vaccines, adjuvants, kits, isolated cells, methods and uses described herein below, characterized in the claims and illustrated by the appended Examples.

### BRIEF DESCRIPTION OF THE DRAWINGS

FIG. 1: Immune regulatory effects of HpbE as compared to effects of commonly used glucocorticosteroids. Data was pooled from at least 2 independent experiments and presented as mean±SEM for MDM from n=3-6 healthy human blood donors. Statistical significance was determined by Friedman test. *p<0.05. (A) Relative gene expression of eicosanoid pathway proteins or IL-10 (qPCR) in human MDM±treatment with HpbE, Dexamethasone (Dex) or Fluticasone propionate (FP) (B) Levels of PGE₂ (EIA) or IL-10 (ELISA) (normalized to levels for HpbE) in human MDM±treatment with HpbE, Dexamethasone (Dex) or Fluticasone propionate (FP) (C) Levels of total 5-LOX or COX products (LC-MS/MS) produced by human MDM±treatment with HpbE, Dexamethasone (Dex) or Fluticasone propionate (FP). (D) Levels of total 5-LOX- or COX products or DiHOMEs (LC-MS/MS) produced by human PMN±treatment with HpbE, Dexamethasone (Dex) or Fluticasone propionate (FP).

FIG. 2: HpbE but not fluticasone propionate induces a shift from pro-inflammatory 5-LOX to regulatory COX and 15-LOX metabolites in macrophages from healthy controls and AERD patients. Levels of eicosanoids (LC-MS/MS) produced by MDM from healthy blood donors or from blood donors suffering from AERD. Data are pooled from at least 2 independent experiments and presented as mean±SEM for MDM from n=3 donors per group. Statistical significance was determined by 2way ANOVA with Bonferroni correction. ***p<0.001.

FIG. 3: Comparison of immuneregulatory effects of L3, L4 and L5 extracts of Hpb. Data are pooled from at least 2 independent experiments and presented as mean±SEM for MDM from n=3-6 healthy human blood donors. Statistical significance was determined by Friedman test. *p<0.05. (A) Levels of PGE₂ or cysLTs (EIA) produced by human MDM±treatment with L3, L4 or L5 extract of HpbE. (B) Levels of IL-1, IL-1β and IL-27 (Bioplex) produced by human MDM±treatment with L3, L4 or L5 extract of HpbE.

FIG. 4: Glutamate dehydrogenase is a major immuneregulatory protein in Hpb L3 larval extract. Data are pooled from at least 2 independent experiments and presented as mean±SEM for MDM from n=3-10 healthy human blood donors. Statistical significance was determined by Friedman test. *p<0.05, **p<0.01, ***p<0.001. (A) Levels of prostanoids (EIA) or IL-10 and IL-1β (ELISA) in human MDM±treatment with HpbE or heat-inactivated HpbE (HpbE 90° C.) or chemotaxis of human PMN±treatment with HpbE or heat-inactivated HpbE (HpbE 90° C.). (B) Levels of IL-10 (ELISA) in human MDM±treatment with HpbE±pretreatment with proteinase K (prot K). (C) Size exclusion chromatogram for fractionation of Hpb L3 extract. (D) Levels of TXB₂ (EIA) or IL-10 (ELISA) in human MDM±treatment with HpbE fractions. (E) Summary of results from mass-spectrometric identification of proteins in active fractions of HpbE. (F) Levels of PGE₂ (EIA) or IL-10 (ELISA) in human MDM±treatment with HpbE±inhibitor of GDH (GDHi, Bithionol, 20 μM). (G) Levels of PGE₂ (LC-MS/MS) or total COX metabolites in human MDM±treatment with HpbE±inhibitor of GDH (GDHi, Bithionol, 20 μM or 100 μM). (H) Levels of PGE₂ (EIA) or IL-10 (ELISA) in human MDM±treatment with HpbE±monoclonal antibody against Hpb GDH (1:10/1:100/1:1000). (I) Levels of PGE₂ (EIA), IL-10 (ELISA) or cysLTs (EIA) in human MDM±treatment with purified recombinant His-tagged Hpb GDH±monoclonal antibody (4F8) against Hpb GDH (1:10).

FIG. 5: New monoclonal antibodies recognize Hpb GDH, but not human GDH. A lysate from E. coli, overexpressing Hpb GDH (lanes 1 and 2 on the left for peptide B/lanes 2 and 3 on the right for peptide A) or a lysate of human MDM (lane 3 on the left for peptide B/lane 1 on the right for peptide A) were probed with newly generated monoclonal antibodies against Hpb GDH (peptides used for immunization are specified above the blots). Clone 4F8 was chosen for further sub cloning and neutralization experiments.

FIG. 6: Infection with the helminth Heligmosomoides polygyrus bakeri (Hpb) or treatment with Hpb larval extract (HpbE) modulates eicosanoid production and type 2 inflammation. (A) Levels of COX and LOX metabolites (LC-MS/MS) in intestinal culture supernatants from naïve mice or mice infected with Hpb (200 L3). (B) Levels of COX and LOX metabolites (LCMS/MS) in peritoneal lavage from naïve mice or mice infected with Hpb. (C) Representative immunofluorescence stainings of COX-2 and HIF-1α or 5-LOX in small intestinal tissue. Dashed lines indicate positioning of Hpb larvae. (D) Top: Experimental model of house dust mite (HDM)-induced allergic airway inflammation and intranasal (i.n) treatment with HpbE; Bottom: BALF cell counts in mice sensitized and challenged with HDM (1 μg/10 μg)±intranasal treatment with HpbE (5 μg). (E) Representative hematoxylin and eosin (H&E)- or Periodic acid-Schiff (PAS) stained lung tissue from mice sensitized to HDM±treatment with HpbE. Scale bar: 100 μm. (F) Levels of 15-HETE (LC-MS/MS) or IL-5, IL-6, eotaxin and RANTES (Bioplex) in BALF from mice sensitized to HDM±treatment with HpbE. Results are pooled from two independent experiments in (A, B, D and F) or representative of stainings performed for two independent experiments in (C and E). Results in (A, B, D and F) are presented as mean±SEM, n=4-10 per group. Statistical significance was determined by 2way ANOVA with Bonferroni correction (A and B) or Kruskal-Wallis test followed by Dunn's multiple comparison test (D and F). *p=0.05, **p=0.01, ***p<0.001.

FIG. 7: HpbE induces a type 2-suppressive eicosanoid profile in macrophages. (A) BALF cell counts or IL-5 levels in mice sensitized to HDM±intranasal transfer of HpbE-conditioned BMDM (wildtype (wt) or PTGS2−/−). (B) Representative H&E stained lung tissue from mice sensitized to HDM±intranasal transfer of untreated or HpbE-conditioned BMDM (wt or PTGS2−/−). Scale bar: 100 μm. Data are pooled from 2 independent experiments and presented as mean±SEM, n=4-11 mice per group. Statistical significance was determined by Kruskal-Wallis test followed by Dunn's multiple comparison test. *p<0.05, **p<0.01, ***p<0.001. (C) Eicosanoid levels (LC-MS/MS) produced by mouse bone marrow macrophages (BMDM) after treatment with Hpb larval extract (HpbE). (D) Relative gene expression of AA-metabolizing enzymes (qPCR) in mouse BMDM treated with HpbE. (E) Heat map showing PUFA metabolites (LC-MS/MS) detected in human monocyte derived macrophages (MDM)±treatment with HpbE. (F) Levels of major bioactive AA metabolites (LC-MS/MS) produced by human MDM±treatment with HpbE. (G) Relative gene expression of eicosanoid pathway proteins (qPCR) in human MDM±treatment with HpbE. Data are presented as mean±SEM, n=8 BMDM from C57BL/6 mice, n=10-15 MDM from healthy human blood donors. Statistical significance was determined by Wilcoxon test. *p<0.05, **p<0.01, ***p<0.001.

FIG. 8. HpbE triggers the production of type 2-suppressive cytokines and modulates M2 polarization of human and mouse macrophages. (A) Levels of IL-10 and IL-1β (ELISA) produced by human MDM±treatment with HpbE. (B) Levels of TNF-α, IL-6, IL-12p70, IL-18, IL-27, IL-33 and CCL17/TARC (Bioplex) produced by human MDM after treatment with HpbE. (C) Levels of IL-10 and IL-1β (Bioplex) produced by mouse BMDM±treatment with HpbE. (D) Gene expression of M2 markers (qPCR) in human MDM±treatment with HpbE. (E) Gene expression of M2 markers (qPCR) in mouse BMDM±treatment with HpbE. Data are presented as mean±SEM, n=3-15 MDM from healthy human blood donors, n=5-8 BMDM from C57BLJ6 mice. Statistical significance was determined by Wilcoxon test. *p<0.05, **p<0.01, ***p<0.001.

FIG. 9. HpbE modulates the COX and LOX metabolism in human granulocytes. (A) Heat map showing PUFA metabolites (LC-MS/MS) detected in mixed human granulocytes±treatment with HpbE. (B) Levels of major bioactive AA metabolites (LC-MS/MS) produced by mixed human granulocytes±treatment with HpbE. (C) Levels of cysteinyl leukotrienes (EIA) produced by purified human eosinophils ±treatment with HpbE. (D) Relative gene expression of AA-metabolizing enzymes (qPCR) in mixed human granulocytes±treatment with HpbE. (E) Levels of LT-synthetic enzymes (LTC4S and LTA4H) (flow cytometry) in human eosinophils ±treatment with HpbE. Data are pooled from at least 3 independent experiments and presented as mean±SEM, n=7-9 mixed granulocytes or purified eosinophils from human blood donors. Statistical significance was determined by Wilcoxon test, *p<0.05, **p<0.01.

FIG. 10: Induction of type 2-suppressive mediators by HpbE is dependent on HIF-1a. (A) Representative immunofluorescence staining of HIF-1α, COX-2, DAPI (cell nuclei) and F4/80 in mouse BMDM±treatment with HpbE. (B) Levels of AA metabolites (LC-MS/MS) in mouse BMDM (wt or HIF-1α floxed/floxed×LysMCre) ±treatment with HpbE. (C) Levels of IL-6, TNFα, IL-1β or IL-10 (Bioplex) in mouse BMDM (wt or HIF-1α floxed/floxed×LysMCre) ±treatment with HpbE. (D) Gene expression of M2 markers (qPCR) in mouse BMDM (wt or HIF-1αfloxed/floxed×LysMCre) ±treatment with HpbE. Data are pooled from at least 2 independent experiments and presented as mean±SEM, n=5-8 BMDM from wt or HIF-1α floxed/floxed×LysMCre mice. Statistical significance was determined by 2way ANOVA. *p<0.05, **p<0.01, ***p<0.001.

FIG. 11. Induction of a type 2-suppressive phenotype in human macrophages is mediated via p-38, HIF-1α and COX. (A) Protein levels of phospho-p38, total p38, COX-2 or β-actin (westernblot) in human MDM±treatment with HpbE. Left: representative blots for human MDM from n=3 blood donors; right: quantification for n=5-9 donors. (B, C) Levels of IL-10 or IL-1β (ELISA) in human MDM±treatment with HpbE±inhibitors of p-38 (VX-702), COX (indomethacin) or HIF-1α (acriflavine). (D) Fold change of PGE2- or LT-synthetic enzymes in human MDM treated with HpbE±inhibitors of p-38 (VX-702), COX (indomethacin) or HIF-1α (acriflavine). Dotted lines indicate levels in untreated cells. Data are pooled from at least 2 independent experiments and presented as mean±SEM, n=6-9 MDM from human blood. Statistical significance was determined by Wilcoxon test for two groups or Friedman test for more than 2 groups. *p<0.05, **p<0.01, ***p<0.001. (E) Suggested mechanism underlying the HpbE-driven modulation of the AA metabolism and type 2 inflammation.

FIG. 12. HpbE and HpbE-treated macrophages inhibit the chemotaxis of human granulocytes in settings of type 2 inflammation. (A) Chemotaxis of granulocytes from AERD patients (n=6) towards nasal polyp secretions ±treatment with HpbE or anti-inflammatory drugs (fluticasone propionate, FP or montelukast, MK). Dashed line depicts basal migration. (B) Levels of chemotactic receptors (CCR3 and CRTH2) (flow cytometry) in human eosinophils ±treatment with HpbE. (C) Chemotaxis of human granulocytes towards a chemokine cocktail±pretreatment with conditioned media from MDM (±HpbE, ±COX-inhibitor indomethacin). Dashed line depicts basal migration. Data are pooled from at least 3 independent experiments and presented as mean±SEM, n=6-8 mixed granulocytes from human blood donors (AERD (A) or healthy (C)). Statistical significance was determined by Wilcoxon test (two groups) or Friedman test (four groups), *p<0.05, **p<0.01.

FIG. 13. 5-lipoxygenase is abundant in tissues of Schistosoma mansoni (Sm) infected mice and larval extract of Sm (SmE) fails to modulate macrophage eicosanoid profiles. (A) Representative immunohistochemical stainings for 5-LOX in naïve lung (left) or in the lung of mice infected with S. mansoni (right). (B) Representative immunohistochemical stainings for 5-LOX in the liver of mice infected with S. mansoni. (C) Eicosanoid levels (LC-MS/MS) produced by human MDM after treatment with larval extracts from Hpb or S. mansoni (SmE). Dashed lines indicate control levels. (D) Levels of IL-10 (ELISA) produced by human MDM±treatment with HpbE or SmE. Dashed line indicates control level. Results are expressed as mean±SEM, n=3-6 per group. Statistical significance was determined by Wilcoxon test (two groups) or Friedman test (more than 2 groups). *p<0.05, **p<0.01.

FIG. 14. Effects of secreted products of adult Hpb (HES), HpbE-associated bacteria, LPS or heattreated HpbE on COX metabolites, cytokines or granulocyte chemotaxis. (A) Levels of prostanoids (LC-MS/MS) or IL-10 (ELISA) in human MDM treated with Hpb larval extract (HpbE) or Hpb excretory secretory products “HES” (10 μg/ml). (B) Relative gene expression of COX pathway enzymes or IL10 (qPCR) in human MDM treated with HpbE or HES. (C) Levels of TXB2 (EIA) or IL-10 (ELISA) produced by human MDM after treatment with HpbE or a homogenate of major bacterial strains present in HpbE. (D) Relative gene expression of COX pathway enzymes or IL10 (qPCR) in human MDM treated with HpbE or a homogenate of major bacterial strains present in HpbE. (E) Levels of prostanoids (LC-MS/MS) produced by MDM treated with HpbE or LPS (60 ng/ml). (F) Levels of prostanoids (EIA) or IL-10 and IL-1β (ELISA) in human MDM±treatment with HpbE or heat-inactivated HpbE (HpbE 90° C.). (G) Chemotaxis of human PMN±treatment with HpbE or heat-inactivated HpbE (HpbE 90° C.).

FIG. 15. HpbE modulates cytokine and eicosanoid production in human PBMCs. (A) Gene expression of type 2 cytokines or IFNG (qPCR) in human PBMCs±treatment with HpbE. (B) Gene expression (qPCR) and protein levels (ELISA) of IL-10 in human PBMCs±treatment with HpbE. (C) Levels of major bioactive AA metabolites (LC-MS/MS) produced by human PBMCs±treatment with HpbE. Data are presented as mean±SEM, n=5-6 PBMCs from healthy human blood donors. Statistical significance was determined by Wilcoxon test. **p<0.01, ***p<0.001.

FIG. 16. Effect of COX-2-, NFKb-, PI3K-, PTEN- or PKA-inhibition on HpbE-driven modulation of cytokines and eicosanoid pathways. (A) Levels of IL-10 or IL-1β (ELISA) produced by human MDM±treatment with HpbE±selective COX-2 inhibitor (10 μM CAY10404). (B) Gene expression of IL10, PGE2-synthetic enzymes or ALOX5 (qPCR) for human MDM treated with HpbE±selective COX-2 inhibitor (CAY10404). (C) Levels of PGE2 (EIA) or IL-10 and IL-1β (ELISA) for human MDM±treatment with HpbE±NFκb inhibitor (5 μM BAY 11-7085). (D) Gene expression of IL10, PGE2-synthetic enzymes or ALOX5 (qPCR) for human MDM treated with HpbE±NFκb inhibitor (BAY 11-7085). (E) Levels of PGE2 (EIA) or IL-10 and IL-1β (ELISA) produced by human MDM after treatment with HpbE±inhibitors of PTEN (250 nM SF1670), PI3K (100 nM Wortmannin) or PKA (10 μM H-89). Data are presented as mean±SEM, MDM from n=5-11 donors. Statistical significance was determined by Wilcoxon test (two groups) or Friedman test (more than 2 groups). *p<0.05, **p<0.01, ***p<0.001.

FIG. 17. Effect of neutralizing antibodies against PRRs (TLR2 and Dectins-1 and -2) or IL-1β on HpbE-driven modulation of eicosanoids and IL-10. (A) Relative gene expression of IL10, PGE2-synthetic enzymes or ALOX5 (qPCR) in human MDM treated with HpbE±blocking antibodies against IL-1β (5 μg/ml) or TLR2 (10 μg/ml). (B) Relative gene expression of IL10, PGE2-synthetic enzymes or ALOX5 (qPCR) in human MDM treated with HpbE±blocking antibodies against dectins-1 or -2 (10 μg/ml). Data are presented as mean±SEM, MDM from n=5-8 donors. Statistical significance was determined by Wilcoxon test (two groups) or Friedman test (more than 2 groups). *p<0.05, **p<0.01, ***p<0.001. Dashed line indicates control level.

### OVERVIEW OF THE SEQUENCE LISTING

As described herein references are made to UniProtKB Accession Numbers (http://www.uniprot.org/, e.g., as available in UniProtKB Release 2018_03 published Mar. 28, 2018).

SEQ ID NO: 1 is the amino acid sequence of Heligmosomoides polygyrus bakeri glutamate dehydrogenase, UniProtKB Accession Number: A0A183FP08.

SEQ ID NO: 2 is the amino acid sequence of Heligmosomoides polygyrus bakeri ferritin; UniProtKB Accession Number: A0A183FLG6.

SEQ ID NO: 3 is the amino acid sequence of Heligmosomoides polygyrus bakeri aspartate aminotransferase; UniProtKB Accession Number: A0A183F107.

SEQ ID NO: 4 is the amino acid sequence of Heligmosomoides polygyrus bakeri tubulin alpha chain; UniProtKB Accession Number: A0A183GTY4.

SEQ ID NO: 5 is the amino acid sequence of Heligmosomoides polygyrus bakeri histone H2B; UniProtKB Accession Number: A0A183FWH9.

### DETAILED DESCRIPTION OF THE INVENTION

### Definitions

As used herein, the term “larval preparation” refers to larvae that were prepared, manufactured, compounded, homogenized and/or purified (e.g., to become cell- and/or cell debris free). Preferably, a larval preparation of the present invention is a cell-free larval preparation (e.g., a L3-larval preparation in a form of a somatic homogenate, e.g., total somatic homogenate of Hpb L3 larvae).

As used herein, the term “Hpb” refers to Heligmosomoides polygyrus bakeri helminths and is equally used herein with the term “Heligmosomoides polygyrus bakeri”. The nematode Heligmosomoides polygyrus (formerly known as Nematospiroides dubius) is a common parasite found in the duodenum and small intestine of woodmice and other rodents (https://parasite.wormbase.org/Helig mosomoides_polygyrus_prjeb1203/Info/Index/). The laboratory strain that has been sequenced was originally isolated from Peromyscus in California (Behnke and Harris, 2010), wherein said laboratory strain is named Heligmosomoides polygyrus bakeri. The laboratory strain is typically maintained as described by Camberis et al., 2003 and is often used to model human helminth infection as it can establish chronic infection in different strains of mice.

As used herein, the terms “L3 larvae”, “L3-developmental stage larva” or “L3-developmental stage Hpb larva” are used interchangeably and refer to Hpb larva that is infective (e.g., capable of infecting mammalian cells) and non-feeding (Camberis et al., 2003), preferably said L3-developmental stage larva is between about 470-570 μm long.

As used herein, the term “extract” refers to the separated phase (often, but not necessarily organic) that contains the material extracted from the other phase. Preferably, the extract of the present invention is a polypeptide or protein extract.

The term “polypeptide” is equally used herein with the term “protein”. Proteins (including fragments thereof, preferably biologically active fragments, and peptides, usually having less than 30 amino acids) comprise one or more amino acids coupled to each other via a covalent peptide bond (resulting in a chain of amino acids). The term “polypeptide” as used herein describes a group of molecules, which, for example, consist of more than 30 amino acids. Polypeptides may further form multimers such as dimers, trimers and higher oligomers, i.e., consisting of more than one polypeptide molecule. Polypeptide molecules forming such dimers, trimers etc. may be identical or non-identical. The corresponding higher order structures of such multimers are, consequently, termed homo- or heterodimers, homo- or hetero-trimers etc. An example for a hetero-multimer is an antibody molecule, which, in its naturally occurring form, consists of two identical light polypeptide chains and two identical heavy polypeptide chains. The terms “polypeptide” and “protein” also refer to naturally modified polypeptides/proteins wherein the modification is affected, e.g., by post-translational modifications like glycosylation, acetylation, phosphorylation and the like. Such modifications are well known in the art.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element.

As used herein, the term “consisting essentially” refers to a larval preparation, polypeptide extract or somatic proteins, in which specific further components can be present, namely those not materially affecting the essential characteristics of the corresponding larval preparation, polypeptide extract or somatic proteins (“consists essentially of”), e.g., said “further components” can be cofactors of Hpb polypeptides.

As used herein, the term “cofactors” refers to organic molecules (cf. coenzymes) or ions (usually metal ions) that are required by an enzyme of its activity. They may be attached either loosely or tightly prosthetic group) to the enzyme. A cofactor binds with its associated protein (apoenzymes), which is functionally inactive, to form the active enzyme (holoenzyme).

As used herein, the term “% identity” refers to the percentage of identical amino acid residues at the corresponding position within the sequence when comparing two amino acid sequences with an optimal sequence alignment as exemplified by the ClustalW or X techniques as available from www.clustal.org, or equivalent techniques. Accordingly, both sequences (reference sequence and sequence of interest) are aligned, identical amino acid residues between both sequences are identified and the total number of identical amino acids is divided by the total number of amino acids (amino acid length). The result of this division is a percent value, i.e. percent identity value/degree.

As used herein, the terms “nucleic acids” or “nucleotide sequences” refer to DNA molecules (e.g. cDNA or genomic DNA), RNA (mRNA), combinations thereof or hybrid molecules comprised of DNA and RNA. The nucleic acids can be double- or single-stranded and may contain double- and single-stranded fragments at the same time. Most preferred are double stranded DNA molecules.

The present invention furthermore provides a nucleic acid vector comprising at least one of the nucleic acid sequences as described herein that encode a polypeptide of the present invention. The vector preferably comprises a promoter under the control of which the above nucleic acid sequences are placed. The vector can be prokaryotic or eukaryotic expression vector, where the recombinant nucleic acid is either expressed alone or in fusion to other peptides or proteins.

The invention also provides a host cell which is transfected with the vector mentioned above. The host cell can be any cell, a prokaryotic cell or a eukaryotic cell and can be used to produce at least parts of a polypeptide of the present invention or fragment or derivative thereof according to the present invention.

An “adjuvant” is a nonspecific stimulant of the immune response.

In another aspect the present invention relates to a pharmaceutical composition comprising as an active ingredient a polypeptide of the present invention or fragment or derivative thereof according to the invention. Said pharmaceutical composition may comprise at least one pharmaceutically acceptable carrier or adjuvant or excipient.

Polypeptides may be provided in pharmaceutically acceptable compositions as known in the art or as listed in a generally recognized pharmacopeia for use in animals, and more particular in humans.

The composition, if desired, can also contain minor amounts of wetting or emulsifying agents, or pH buffering agents. These compositions can take the form of solutions, suspensions, emulsion, tablets, pills, capsules, powders, sustained-release formulations and the like.

The compositions of the invention can be formulated as neutral or salt forms.

Pharmaceutically acceptable salts include, but are not limited to those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The dosage amounts and frequencies of administration are encompassed by the terms therapeutically effective and prophylactically effective. The dosage and frequency of administration further will typically vary according to factors specific for each patient depending on the specific therapeutic or prophylactic agents administered, the type of disease, the route of administration, as well as age, body weight, response, and the past medical history of the patient. Suitable regimens can be selected by one skilled in the art. As used herein, the term “therapeutically effective amount” refers to an amount of the therapeutic active component or agent which is sufficient to treat or ameliorate a disease or disorder, to delay the onset of a disease or which provides any therapeutical benefit in the treatment or management of a disease.

As used herein, the term “treating” and “treatment” refers to administering to a subject a therapeutically effective amount of a pharmaceutical composition according to the invention. A “therapeutically effective amount” refers to an amount of the pharmaceutical composition or the antibody which is sufficient to treat or ameliorate a disease or disorder, to delay the onset of a disease or to provide any therapeutical benefit in the treatment or management of a disease.

As used herein, the term “prophylaxis” refers to the use of an agent for the prevention of the onset of a disease or disorder. A “prophylacticly effective amount” defines an amount of the active component or pharmaceutical agent sufficient to prevent the onset or recurrence of a disease.

As used herein, the terms “disorder” and “disease” are used interchangeably to refer to a condition in a subject.

In a preferred embodiment of the invention the diagnostic composition as described herein is for the detection and diagnosis of any disease or disorder, especially a disease selected from the group consisting of: chronic respiratory disease, steroid resistant airway inflammation, aspirin-exacerbated respiratory disease (AERD), nasal polyps, cystic fibrosis (CF), allergic rhino-conjunctivitis, atopic dermatitis, autoimmune disease, inflammatory disease, chronic inflammatory disease, rhinitis, diabetes; bronchitis, chronic bronchitis, mucopurulent chronic bronchitis, emphysema, MacLeod syndrome, panlobular emphysema, centrilobular emphysema, chronic obstructive pulmonary disease (COPD), chronic obstructive pulmonary disease with acute lower respiratory infection, chronic obstructive pulmonary disease with acute exacerbation, asthma, predominantly allergic asthma, atopic asthma, extrinsic allergic asthma, non-allergic asthma, idiosyncratic asthma, intrinsic nonallergic asthma, mixed asthma, asthmatic bronchitis, late-onset asthma, status asthmaticus, acute severe asthma, bronchiectasis, nasal polyps, cystic fibrosis (CF), allergic rhino-conjunctivitis, atopic dermatitis, autoimmune or inflammatory disease, allergy.

Exemplary autoimmune diseases of the present invention include immune thrombocytopenia, systemic lupus erythematosus, pernicious anemia, Addison's disease, diabetis type 1, rheumatoid Arthritis, Sjogren's syndrome, dermato-myositis, multiple sclerosis, myasthenia gravis, Reiter's syndrome, Graves' disease, Pemphigus vulgaris and bullosus, autoimmune hepatitis, ulcerative colitis, cold agglutinin disease, autoimmune peripheral neuropathy, but are not limited to these.

Examples of the inflammatory diseases of the present invention include: acne vulgaris, asthma, autoimmune diseases, autoinflammatory diseases, celiac disease, chronic prostatitis, colitis, diverticulitis, glomerulonephritis, hidradenitis suppurativa, hypersensitivities, inflammatory bowel diseases, interstitial cystitis, Lichen planus, mast cell activation syndrome, mastocytosis, otitis, pelvic inflammatory disease, reperfusion injury, rheumatic fever, rheumatoid arthritis, rhinitis, sarcoidosis, transplant rejection, vasculitis.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range.

The following detailed description refers to the accompanying Examples that show, by way of illustration, specific details and embodiments, in which the invention may be practised. These embodiments are described in sufficient detail to enable those skilled in the art to practice the invention. Other embodiments may be utilized such that structural, logical, and eclectic changes may be made without departing from the scope of the invention. Various aspects of the present invention described herein are not necessarily mutually exclusive, as aspects of the present invention can be combined with one or more other aspects to form new embodiments of the present invention.

In the course of the present invention Hpb proteins and preparations were identified and isolated that are capable of broadly modulating inflammatory responses, e.g., by (i) suppressing the production of LTs, (ii) inducing the production of anti-inflammatory mediators (prostaglandin E2, IL-10) and (iii) reducing granulocyte recruitment and activation. Thus, the identified Hpb proteins and preparations of the present invention target several key mechanisms of chronic airway inflammation at the same time (i.e. simultaneously). None of the currently available anti-inflammatory drugs (e.g. glucocorticosteroids, LT receptor antagonist (LTRA) (e.g., Montelukast, mepolizumab, omalizumab) shows a similar profile of activities. Accordingly, “modulating the innate mammalian immune system” as used herein may relate to (i) suppressing the production of LTs, (ii) inducing the production of anti-inflammatory mediators (such as prostaglandin E2, IL-10, IL-27) and/or (iii) reducing granulocyte recruitment and activation. Methods for assessing these features are known to a person skilled in the art and exemplified in the examples.

It was also shown that Hpb proteins and preparations of the present invention could suppress airway inflammation in mice in vivo if applied topically, which represents an advantage compared to systemic treatment with current immunomodulatory proteins such as monoclonal antibodies (e.g., mepolizumab, omalizumab). Due to the capacity to induce PGE2 and IL-10 Hpb proteins and preparations of the present invention could potentially be used to suppress TH2 differentiation and are thus promising candidates for improving the efficacy of allergen specific immunotherapy.

The LT-suppressive and overall anti-inflammatory potential of the Hpb proteins and preparations of the present invention is surprising as Hpb nematode larvae are usually assumed to trigger eosinophilia and LT production (e.g., Patnode, 2014). Therefore, the present invention harnesses a novel, unique and unexpected potential of immunomodulatory Hpb proteins and preparations obtainable from Hpb nematode to supress several key inflammatory events, e.g., in asthma and nasal polyps. Hpb proteins and preparations of the present invention do not only impact on the 5-lipoxygenase pathway to suppress LT production, but also induce regulatory factors such as PGE2, which has important, therapy-relevant anti-inflammatory effects in the airways, including the suppression of remodeling (Stumm et al. 2011), efficient bronchodilation (e.g., better than Salbutamol) (FitzPatrick M et al. 2014). Moreover, the Hpb proteins and preparations of the present invention reduce the expression of major chemotactic receptors on eosinophils, an effect, which has not been described for any of the current standard treatments. Indeed, Hpb proteins and preparations of the present invention reduced granulocyte migration ex vivo (e.g., in patient cells) and in vivo (e.g., in murine asthma model). This suggests efficacy against tissue infiltration with neutrophils and eosinophils (a hallmark of nasal polyps and severe asthma). The parasitic nematode Hpb, which is the source of the immunomodulatory proteins and preparations of the present invention, does not express toxic molecules, which would harm the host. Thus, the novel Hpb proteins and preparations of the present invention are unlikely to show considerable toxicity, particularly when applied topically. Hpb proteins and preparations of the present invention are also unlikely to pass the blood brain barrier (e.g. as Montelukast) or show profound metabolic side effects such as Cushings-syndrome (e.g. as glucocorticosteroids might do) as rodents do not show these symptoms during infection with H. polygyrus. Indeed, helminths have even been shown to have beneficial effects on diabetes (e.g., Mishra et al. 2013). Taken together, Hpb proteins and preparations of the present invention show a broader immunomodulatory profile than current anti-inflammatory treatments and fewer adverse side effects. In addition to their potential as a new anti-inflammatory therapy, Hpb proteins and preparations of the present invention could be used as new adjuvants in allergen specific immunotherapy. This application is based on the potential of Hpb-induced PGE2 and IL-10 to suppress TH2 cell differentiation and survival (Khan 1995, Coomes et al. 2017).

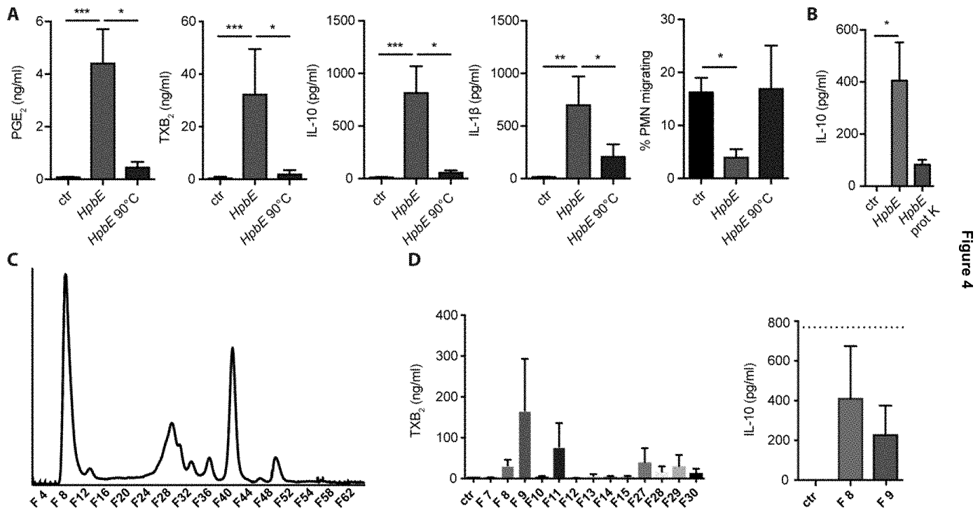

Although larval preparations of Hpb have already been disclosed, e.g. in DE 10163602, US 2010/303721 or WO 2018/02523, none of these disclosures explicitly provides an incentive to use cell-free larval preparations of Hpb obtained from cells of the L3-developmental stage. In contrast, WO 2018/02523 even suggests using L4 larvae of Hpb. However, the inventors surprisingly found that L3 larval extracts have a higher efficacy in modulating the immune system, e.g. reducing levels of cysLTs or inducing PGE₂, IL-10, IL-1β and IL-27 as shown in Example 2.3 FIG. 3, than L4 and L5 larval extracts (somatic preparations). Indeed, L4 such as suggested by WO 2018/02523 and L5 stage extracts of Hpb fail to induce type 2-suppressive mediators. Thus, the technical effect of using L3 stage extracts instead of L4 or L5 stage extracts provides improved immunomodulatory effects, e.g. increasing immune-suppressive factors such as PGE₂, IL-10, IL-1β or IL-27 and reducing immune-stimulatory factors such as CXCL10 or cysLTs.

Based on the above at least the following advantages of the present invention are contemplated over molecules known from the prior art and methods based thereon:

1) Production costs of the Hpb proteins and preparations of the present invention are relatively low, e.g., if compared to the production costs of the far more complex antibody molecules.

2) Hpb proteins and preparations of the present invention have a much lower immunogenicity, e.g., if compared to current “biologicals” (e.g., antibodies) as the identified Hpb proteins have human protein homologues.

3) As the identified Hpb proteins and preparations of the present invention are capable of mainly targeting phagocytic cells and acting intracellularly, they may also be encapsulated to further reduce the risk of an adverse immunogenic reaction.

4) Hpb proteins and preparations of the present invention are active when applied topically (i.e., they are suitable for topical administration), e.g., to the airways of subjects in need thereof during the airway inflammation.

5) Hpb proteins and preparations of the present invention are not only capable of supressing LT production by myeloid cells (e.g., including eosinophils), but simultaneously capable of inducing anti-inflammatory mediators (e.g., PGE2, IL-10), an effect, which is not achieved by current treatments.

6) Hpb proteins and preparations of the present invention are particularly suitable for immunomodulation in a mammalian host environment (e.g., human) as Hpb helminths co-evolved with their mammalian host, which resulted in the development of immunomodulatory compounds that are non-toxic to mammal hosts (e.g., there is no hepatotoxic effect associated with them), but are capable of modulating a variety of mechanisms to supress inflammatory immune response, thus allowing for both host and parasite survival.

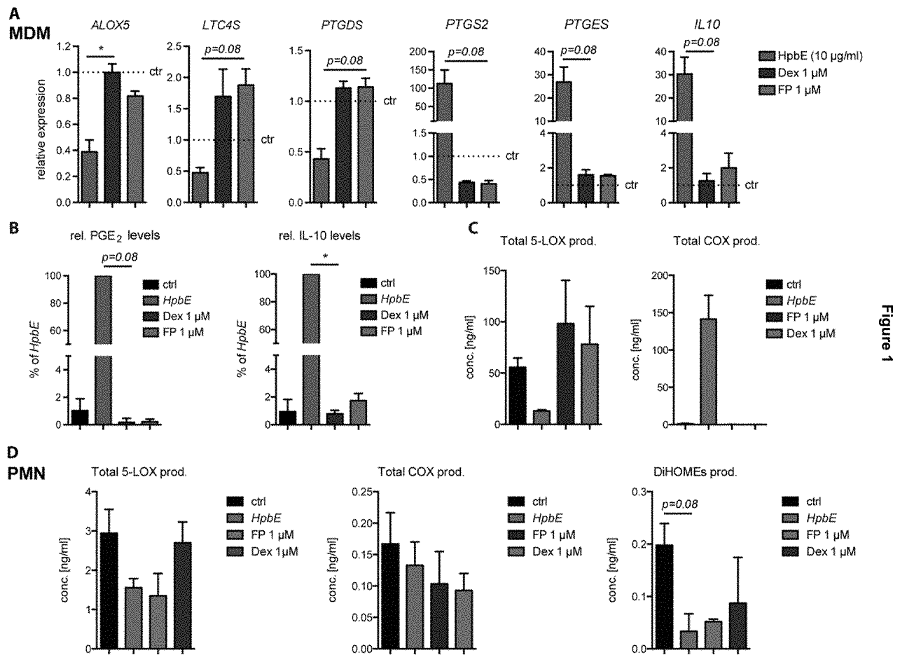

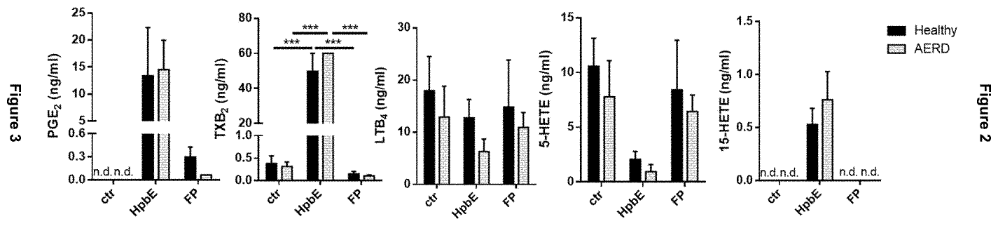

7) Hpb proteins and preparations of the present invention have a “natural” Hpb origin, which may result in a better acceptance of the treatment methods based on Hpb proteins and preparations of the present invention by patients, who are often sceptical about the use of glucocorticosteroids, blood brain barrier crossing drugs such as LTRAs or multiple invasive sinus surgeries. In addition and as shown in FIG. 1 and Example 2.1, they have also a higher efficacy (related to multiple immuneregulatory effects, which are beneficial in allergy, asthma or similar diseases).

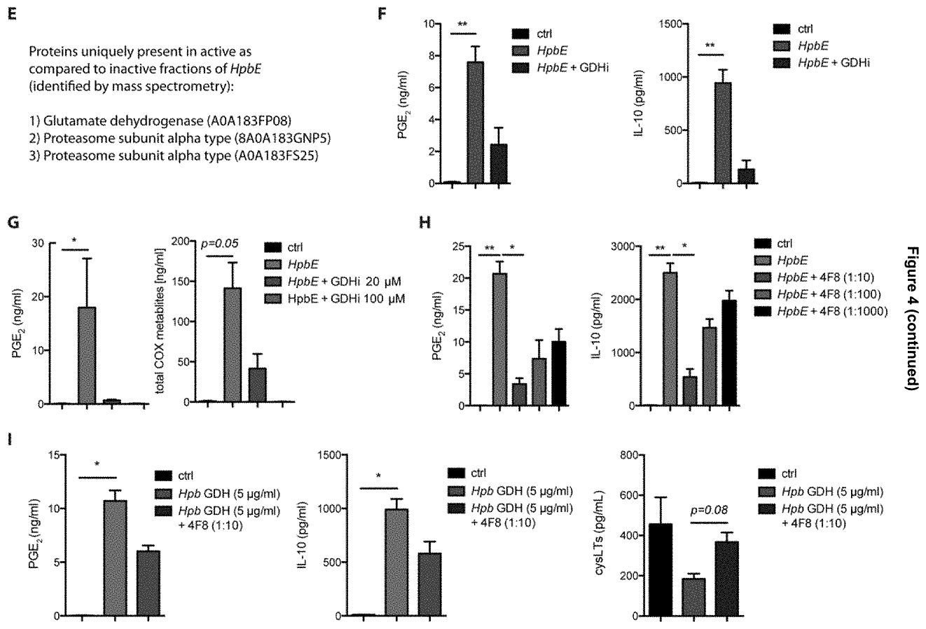

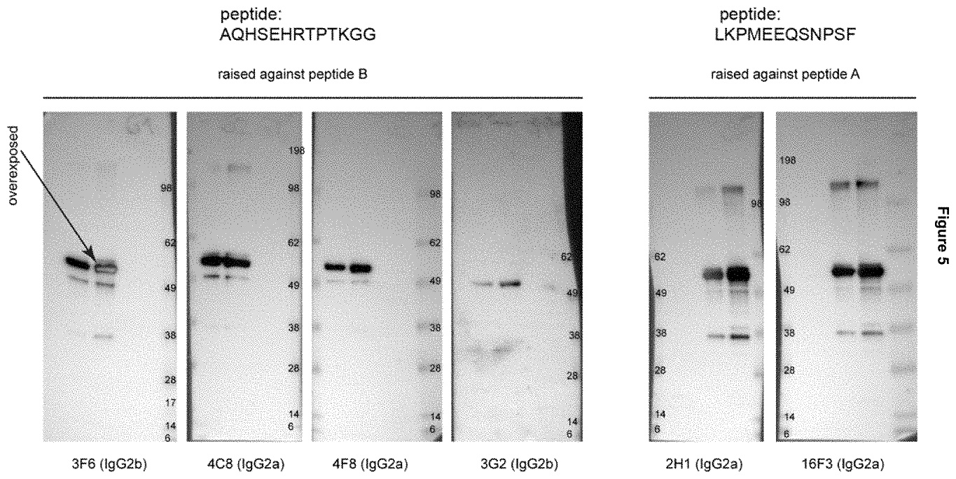

The glutamate dehydrogenase (GDH), e.g. as defined in UniProtKB Accession Number A0A183FP08 (e.g., SEQ ID NO: 1) is a component of L3 larval extracts of Hpb as shown, e.g. in Example 1.3. The inventors could show that GDH alone has comparable immunomodulatory effects to that of L3 larval preparations (see Example 2.4, FIG. 4). Accordingly, the present invention relates to a polypeptide for use as a medicament, wherein said polypeptide is capable of modulating the innate mammalian immune system and is at least 60% or more (e.g., at least 65%, at least 70%, at least 75%, at least 80%, at least 85%, at least 90%, at least 95%, at least 96%, at least 97%, at least 98%, at least 99% or 100%) identical to a polypeptide obtainable from L3-developmental stage larva of Heligmosomoides polygyrus bakeri (Hpb) helminths, wherein the polypeptide is Hpb glutamate dehydrogenase polypeptide, said glutamate dehydrogenase polypeptide having UniProtKB Accession Number: A0A183FP08 or the amino acid sequence as depicted in SEQ ID NO: 1. Preferably said polypeptide is capable of inducing of anti-inflammatory mediators as defined herein and/or preferably said polypeptide has EC 1.4.1.2 or EC 1.4.1.3 or EC 1.4.1.4 enzymatic activity.

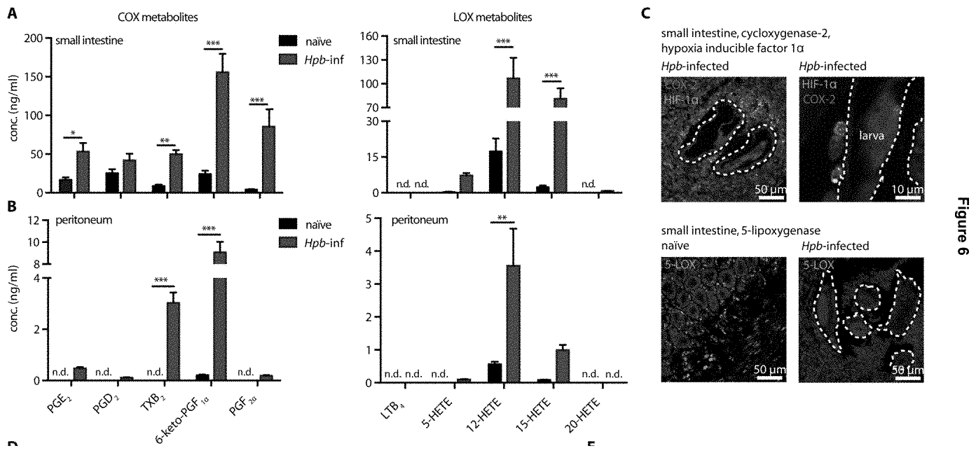

For their studies, the inventors also made use of novel anti-GDH antibodies (see Example 2.4 and FIG. 5). These antibodies bind to a peptide having the amino acid sequence AQHSEHRTPTKGG (SEQ ID NO: 6) (antibodies 3F6, 4C8, 4F8, 3G2) or a peptide having the amino acid sequence LKPMEEQSNPSF (SEQ ID NO: 7) (antibodies 2H1, 16F3). Accordingly, the present invention relates to an antibody that binds to a peptide having the amino acid sequence AQHSEHRTPTKGG (SEQ ID NO: 6) or a portion thereof. In a related embodiment, the present invention relates to an antibody that binds to a peptide having the amino acid sequence LKPMEEQSNPSF (SEQ ID NO: 7) or a portion thereof. The inventors could further show in Example 2.4 and FIG. 5 that these antibodies do not bind to mammalian (human/mouse) GDH. Thus, the inventors found a new tool for studying the uptake, localization and function of Hpb GDH in vivo and in target cells in vitro. Accordingly, the antibodies of the present invention preferably are not cross-reactive and/or do not bind to mammalian, preferably human and/or mouse, GDH. The feature “not cross-reactive and/or do not bind to mammalian, preferably human and/or mouse, GDH” as used within the context of the antibodies of the present invention was shown by the Inventors by performing a Western Blot analysis of human MDM (monocyte-derived macrophage) lysate comprising GDH and a lysate of E. coli that overexpressed Hpb GDH (see FIG. 5). Thus, the antibodies of the present invention preferably are not cross-reactive with and/or do not bind to mammalian, preferably human and/or mouse, GDH, wherein the cross-reactivity and/or binding to is analysed by Western Blot analysis of a lysate comprising human and/or mouse GDH and a lysate comprising Hpb GDH, wherein preferably the antibody does not show a signal for the lysate comprising human and/or mouse GDH but shows a signal for the lysate comprising Hpb GDH.

The invention is also characterized by the following items:
1. A larval preparation (e.g., a cell-free preparation) of Heligmosomoides polygyrus bakeri (Hpb) helminths (e.g., a somatic homogenate, e.g., total somatic homogenate of Hpb L3 larvae), wherein said larval preparation is obtainable (e.g., obtained) from cells of the L3-developmental stage larva of said Hpb helminths, wherein said larval preparation is capable of modulating the innate mammalian immune system.
2. The larval preparation according to any one of preceding items, wherein said larval preparation is predominantly (e.g., more than 50% of its overall effect on the mammalian immune system) capable of modulating the innate mammalian immune system over (e.g., compared to its effect on the adaptive mammalian immune system) the adaptive mammalian immune system (e.g., said larval preparation is capable of having a greater modulating effect on the innate mammalian immune system than on the adaptive mammalian immune system).
3. The larval preparation according to any one of preceding items, wherein said L3-developmental stage larva is an infective (e.g., capable of infecting mammalian cells) non-feeding larva, preferably said L3-developmental stage larva is between about 470-570 μm long.
4. The larval preparation according to any one of preceding items, wherein said larval preparation comprises a polypeptide extract obtainable (e.g., obtained) from cells of the L3-developmental stage larva of said Hpb helminths, preferably said polypeptide extract consisting essentially of polypeptides (e.g., oligomeric polypeptides and/or monomeric polypeptides) with molecular weight of 3 or more kDa; further preferably said polypeptide extract consisting essentially of polypeptides, wherein said polypeptides including oligomeric and/or monomeric polypeptides, with molecular weight of monomeric polypeptides in the range of about 3-70 kDa, most preferably said polypeptide extract consisting essentially of polypeptides, wherein said polypeptides including oligomeric and/or monomeric polypeptides, with molecular weight of monomeric polypeptides in the range of about 9-60 kDa.
5. The larval preparation according to any one of preceding items, wherein said larval preparation comprises a solution of somatic proteins obtainable (e.g., obtained) from cells of the L3-developmental stage larva of said Hpb helminths, preferably said somatic proteins consisting essentially of polypeptides (e.g., oligomeric polypeptides and/or monomeric polypeptides) with molecular weight of 3 or more kDa; further preferably said solution is aqueous, most preferably said somatic proteins consisting essentially of polypeptides, wherein said polypeptides including oligomeric and/or monomeric polypeptides, with molecular weight of monomeric polypeptides in the range of about 3-70 kDa, further most preferably said somatic proteins consisting essentially of polypeptides, wherein said polypeptides including oligomeric and/or monomeric polypeptides, with molecular weight of monomeric polypeptides in the range of about 9-60 kDa.
6. The larval preparation according to any one of preceding items, wherein said larval preparation consists of an aqueous solution of a protein extract obtained from whole-larval homogenate (e.g., somatic whole larval homogenate) of L3-developmental stage larva of Hpb helminths, preferably said polypeptide extract consisting essentially of polypeptides (e.g., oligomeric polypeptides and/or monomeric polypeptides) with molecular weight of 3 or more kDa; further preferably said protein extract consisting essentially of polypeptides including oligomeric and/or monomeric polypeptides with molecular weight of monomeric polypeptides in the range of about 3-70 kDa, most preferably said polypeptide extract consisting essentially of polypeptides including oligomeric and/or monomeric polypeptides with molecular weight of monomeric polypeptides in the range of about 9-60 kDa.
7. The larval preparation according to any one of preceding items, wherein said larval preparation consisting essentially of polypeptides obtainable (e.g., obtained) from cells of the L3-developmental stage larva, preferably said larval preparation does not comprise polypeptides obtained from cells of non-L3 developmental stage of said Hpb helminths, further preferably said larval preparation does not comprise polypeptides obtained from cells of either adult Hpb helminths or L4 larval developmental stage of said Hpb helminths, most preferably said larval preparation consisting of polypeptides obtained from cells of the L3-developmental stage Hpb larva.
8. The larval preparation according to any one of preceding items, wherein said larval preparation is capable of one or more of the following:
   i) targeting phagocytic cells of said mammalian immune system; preferably said phagocytic cells: macrophages, neutrophils or dendritic cells (DC);
   ii) modifying the activation of macrophages and/or granulocytes of mammalian immune system, preferably said granulocytes are eosinophils;
   iii) acting intracellularly;
   iv) modifying the activation of one or more of the leukotriene pathway of mammalian immune system;
   v) decreasing the number of eosinophils and/or inhibiting the migration of granulocytes into tissue of said mammalian immune system;
   vi) inhibiting tissue infiltration with neutrophils and/or eosinophils in mammals;
   vii) binding to an iron atom associated with a mammalian arachidonate 5-lipoxygenase (5-LOX, e.g., a human LOXS having UniProtKB Accession Number: P09917) enzyme and/or iron atom associated with a mammalian cyclooxygenase (COX, e.g., a human Prostaglandin G/H synthase 2 (PTGS2) having UniProtKB Accession Number: P35354; or human cytochrome c oxidase subunit 1 having UniProtKB Accession Number: P00395) enzyme, preferably said mammalian enzyme is a human enzyme.
   viii) reducing the expression and/or inhibiting one or more of the following:
      (a) chemotactic receptors (e.g., human CXC chemokine receptors, human CC chemokine receptors, e.g., C—C chemokine receptor type 3 (UniProtKB Accession Number: P51677), human C chemokine receptors, human CX₃C chemokine receptors or human formyl peptide receptors (FPR), e.g., having UniProtKB Accession Number: P21462, P25090 or P25089);
      (b) cysteinyl leukotriene receptor 1 (CYSLTR1, e.g., having UniProtKB Accession Number: Q9Y271), preferably said CYSLTR1 is expressed by eosinophils;
      (c) leukotriene C4 synthase (LTC₄ synthase, e.g., having UniProtKB Accession Number: Q16873);
      (d) arachidonate 5-lipoxygenase (5-LOX, e.g., having UniProtKB Accession Number: P09917).
9. The larval preparation according to any one of preceding items, wherein said larval preparation is capable of one or more of the following:
   i) suppressing production of leukotrienes (e.g., eicosanoid inflammatory mediators); preferably said leukotrienes are produced by myeloid cells including eosinophils;
   ii) inducing of anti-inflammatory mediators, preferably said anti-inflammatory mediators comprise prostaglandin E2 (PGE2 or (5Z,13E,15S)-11a,15-dihydroxy-9-oxoprosta-5,13-dien-1-oic acid) and/or interleukin 10 (IL-10, e.g., having UniProtKB Accession Number: P22301);
   iii) reducing granulocyte recruitment and/or activation;
   iv) inhibiting of arachidonate 5-lipoxygenase (5-LOX, e.g., having UniProtKB Accession Number: P09917) having EC 1.13.11.34 enzymatic activity,
   v) simultaneously capable of:
      a) (i) and (ii); and/or
      b) (i), (ii) and (iii); and/or
      c) (i), (ii), (iii) and (iv).
10. The larval preparation according to any one of preceding items, wherein said larval preparation comprises one or more of the following polypeptides:
   i) a polypeptide, which is at least 60% or more (e.g., at least 65%, at least 70%, at least 75%, at least 80%, at least 85%, at least 90%, at least 95%, at least 96%, at least 97%, at least 98%, at least 99% or 100%) identical to Hpb glutamate dehydrogenase polypeptide, said glutamate dehydrogenase polypeptide having UniProtKB Accession Number: A0A183FP08 (e.g., SEQ ID NO: 1); preferably said polypeptide having EC 1.4.1.2 or EC 1.4.1.3 or EC 1.4.1.4 enzymatic activity; further preferably said polypeptide is capable of inducing of anti-inflammatory mediators according to any one of preceding items;
   ii) a polypeptide, which is at least 60% or more (e.g., at least 65%, at least 70%, at least 75%, at least 80%, at least 85%, at least 90%, at least 95%, at least 96%, at least 97%, at least 98%, at least 99% or 100%) identical to Hpb ferritin polypeptide, said ferritin polypeptide having UniProtKB Accession Number A0A183FLG6 (e.g., SEQ ID NO: 2) or A0A183FDM1, preferably said polypeptide having EC 1.16.3.1 enzymatic activity; further preferably said polypeptide is capable of suppressing production of leukotrienes according to any one of preceding items;
   iii) a polypeptide, which is at least 60% or more (e.g., at least 65%, at least 70%, at least 75%, at least 80%, at least 85%, at least 90%, at least 95%, at least 96%, at least 97%, at least 98%, at least 99% or 100%) identical to Hpb aspartate aminotransferase polypeptide, said aspartate aminotransferase polypeptide having UniProtKB Accession Number: A0A183F107 (e.g., SEQ ID NO: 3), preferably said polypeptide having EC 2.6.1.1 enzymatic activity;
   iv) a polypeptide, which is at least 60% or more (e.g., at least 65%, at least 70%, at least 75%, at least 80%, at least 85%, at least 90%, at least 95%, at least 96%, at least 97%, at least 98%, at least 99% or 100%) identical to Hpb tubulin alpha chain polypeptide; said tubulin alpha chain polypeptide having UniProtKB Accession Number: A0A183GTY4 (e.g., SEQ ID NO: 4), A0A183F2N5, A0A183FGY7, A0A183FJ38 or A0A183G7U3;
   v) a polypeptide, which is at least 60% or more (e.g., at least 65%, at least 70%, at least 75%, at least 80%, at least 85%, at least 90%, at least 95%, at least 96%, at least 97%, at least 98%, at least 99% or 100%) identical to Hpb histone H2B polypeptide; said histone H2B polypeptide having UniProtKB Accession Number: A0A183F3C5, A0A183FWH9 (e.g., SEQ ID NO: 5), A0A183GMUO or A0A183GQR4;
   vi) a polypeptide, which is at least 60% or more (e.g., at least 65%, at least 70%, at least 75%, at least 80%, at least 85%, at least 90%, at least 95%, at least 96%, at least 97%, at least 98%, at least 99% or 100%) identical to a Hpb polypeptide selected from the group consisting of:
      a. a proteasome subunit;
      b. a TCP-I/cpn60 chaperonin family protein;
      c. a myosin domain (e.g. N-terminal SH3-like domain, head domain),
      d. a Vitamin B12 binding domain,
      e. an Immunoglobulin I-set domain,
      f. a Peptidase M17/Leucin Aminopeptidase,
      g. a Glycosyl hydrolases family 2, sugar binding domain,
      h. an A-macroglobulin complement component,
      i. an Enolase, N-terminal domain,
      j. an ERAP1-like C-terminal domain,
      k. a ribosomal L5P family C-terminus,
      l. an Acetyl-CoA hydrolase/transferase N-terminal domain,
      m. a Cys/Met metabolism PLP-dependent enzyme,
      n. a Fructose bisphosphate aldolase,
      o. an Aminopeptidase I zinc metalloprotease (M18) and
      p. an Cysteine-rich secretory protein family member;
   vii) a polypeptide as in defined (i)-(vi), wherein said polypeptide is orthologous or paralogous to the Hpb polypeptide as defined in (i)-(vi);
   viii) a polypeptide as in defined (i)-(vi), wherein said polypeptide is a fragment of the Hpb polypeptide as defined in (i)-(vi), preferably said fragment having at least 20% or more (e.g., at least 30%, at least 40%, at least 50%, at least 60%, at least 70%, at least 80%, at least 90%, or 100%) of the polypeptide sequence of the Hpb polypeptide as defined in (i)-(vi);
   ix) combinations of (i)-(viii), preferably a combination of (i) and (ii).
11. The larval preparation according to any one of preceding items, wherein said larval preparation comprises one or more of the following polypeptides, wherein said one or more polypeptides is selected from the group consisting of:
   i) Hpb glutamate dehydrogenase polypeptide, said glutamate dehydrogenase polypeptide having UniProtKB Accession Number: A0A183FP08; preferably said polypeptide having EC 1.4.1.2 or EC 1.4.1.3 or EC 1.4.1.4 enzymatic activity; further preferably said polypeptide is capable of inducing of anti-inflammatory mediators according to any one of preceding items;
   ii) Hpb ferritin polypeptide, said ferritin polypeptide having UniProtKB Accession Number A0A183FLG6 or A0A183FDM1; preferably said polypeptide having EC 1.16.3.1 enzymatic activity; further preferably said polypeptide is capable of suppressing production of leukotrienes according to any one of preceding items;
   iii) Hpb aspartate aminotransferase polypeptide, said aspartate aminotransferase polypeptide having UniProtKB Accession Number: A0A183F107;
   iv) Hpb tubulin alpha chain polypeptide, said tubulin alpha chain polypeptide having UniProtKB Accession Number: A0A183GTY4, A0A183F2N5, A0A183FGY7, A0A183FJ38 or A0A183G7U3;
   v) Hpb histone H2B polypeptide, said histone H2B polypeptide having UniProtKB Accession Number: A0A183F3C5, A0A183FWH9, A0A183GMUO or A0A183GQR4;
   vi) Hpb proteasome subunit;
   vii) Hpb TCP-I/cpn60 chaperonin family protein;
   viii) Hpb myosin domain (e.g. N-terminal SH3-like domain, head domain),
   ix) Hpb Vitamin B12 binding domain,
   x) Hpb Immunoglobulin I-set domain,
   xi) Hpb Peptidase M17/Leucin Aminopeptidase,
   xii) Hpb Glycosyl hydrolases family 2, sugar binding domain,
   xiii) Hpb A-macroglobulin complement component,
   xiv) Hpb Enolase, N-terminal domain,
   xv) Hpb ERAP1-like C-terminal domain,
   xvi) Hpb ribosomal L5P family C-terminus,
   xvii) Hpb Acetyl-CoA hydrolase/transferase N-terminal domain,
   xviii) Hpb Cys/Met metabolism PLP-dependent enzyme,
   xix) Hpb Fructose bisphosphate aldolase,
   xx) Hpb Aminopeptidase I zinc metalloprotease (M18) or
   xxi) Hpb Cysteine-rich secretory protein family member;
   xxii) the polypeptide as in defined (i)-(xxi), wherein said polypeptide is a fragment of the Hpb polypeptide as in defined (i)-(xxi), preferably said fragment having at least 20% or more (e.g., at least 30%, at least 40%, at least 50%, at least 60%, at least 70%, at least 80%, at least 90%, or 100%) of the polypeptide sequence of the Hpb polypeptide as defined in (i)-(xxi);
   xxiii) combinations of (i)-(xxii), preferably a combination of (i) and (ii).
12. The larval preparation according to any one of preceding items, wherein said larval preparation is one or more of the following:
   i) is encapsulated;
   ii) is non-toxic to a mammal (e.g., no hepatotoxic effects), further preferably said mammal is a human;
   iii) is not capable to pass through mammalian blood-brain barrier.
13. The larval preparation according to any one of preceding items, wherein said mammalian innate immune system is the human innate immune system.
14. The larval preparation according to any one of preceding items, wherein said adaptive mammalian immune system is the human adaptive immune system.
15. A polypeptide for use as a medicament, wherein said polypeptide is capable of modulating the innate mammalian immune system and is at least 60% or more (e.g., at least 65%, at least 70%, at least 75%, at least 80%, at least 85%, at least 90%, at least 95%, at least 96%, at least 97%, at least 98%, at least 99% or 100%) identical to a polypeptide obtainable from L3-developmental stage larva of Heligmosomoides polygyrus bakeri (Hpb) helminths selected from the group consisting of:
   i) Hpb glutamate dehydrogenase polypeptide, said glutamate dehydrogenase polypeptide having UniProtKB Accession Number: A0A183FP08 (e.g., SEQ ID NO: 1); preferably said polypeptide is capable of inducing of anti-inflammatory mediators according to any one of preceding items
   ii) Hpb ferritin polypeptide, said ferritin polypeptide having UniProtKB Accession Number A0A183FLG6 (e.g., SEQ ID NO: 2) or A0A183FDM1; preferably said polypeptide is capable of suppressing production of leukotrienes according to any one of preceding items;
   iii) Hpb aspartate aminotransferase polypeptide, said aspartate aminotransferase polypeptide having UniProtKB Accession Number: A0A183F107 (e.g., SEQ ID NO: 3);
   iv) Hpb tubulin alpha chain polypeptide, said tubulin alpha chain polypeptide having UniProtKB Accession Number: A0A183GTY4 (e.g., SEQ ID NO: 4), A0A183F2N5, A0A183FGY7, A0A183FJ38 or A0A183G7U3;
   v) Hpb histone H2B polypeptide, said histone H2B polypeptide having UniProtKB Accession Number: A0A183F3C5, A0A183FWH9 (e.g., SEQ ID NO: 5), A0A183GMUO or A0A183GQR4;
   vi) Hpb proteasome subunit;
   vii) Hpb TCP-I/cpn60 chaperonin family protein;
   viii) Hpb myosin domain (e.g. N-terminal SH3-like domain, head domain),
   ix) Hpb Vitamin B12 binding domain,
   x) Hpb Immunoglobulin I-set domain,
   xi) Hpb Peptidase M17/Leucin Aminopeptidase,
   xii) Hpb Glycosyl hydrolases family 2, sugar binding domain,
   xiii) Hpb A-macroglobulin complement component,
   xiv) Hpb Enolase, N-terminal domain,
   xv) Hpb ERAP1-like C-terminal domain,
   xvi) Hpb ribosomal L5P family C-terminus,
   xvii) Hpb Acetyl-CoA hydrolase/transferase N-terminal domain,
   xviii) Hpb Cys/Met metabolism PLP-dependent enzyme,
   xix) Hpb Fructose bisphosphate aldolase,
   xx) Hpb Aminopeptidase I zinc metalloprotease (M18);
   xxi) Hpb Cysteine-rich secretory protein family member;
   xxii) the polypeptide as in defined (i)-(xxi), wherein said polypeptide is orthologous or paralogous to the Hpb polypeptide as defined in (i)-(xxi);
   xxiii) the polypeptide as in defined (i)-(xxii), wherein said polypeptide is a fragment of the Hpb polypeptide as in defined (i)-(xxii), preferably said fragment having at least 20% or more (e.g., at least 30%, at least 40%, at least 50%, at least 60%, at least 70%, at least 80%, at least 90%, or 100%) of the polypeptide sequence of the Hpb polypeptide as defined in (i)-(xxii).
16. The polypeptide for use as a medicament according to any one of preceding items, wherein the polypeptide is Hpb glutamate dehydrogenase polypeptide, said glutamate dehydrogenase polypeptide having UniProtKB Accession Number: A0A183FP08 (e.g., SEQ ID NO: 1); preferably said polypeptide is capable of inducing of anti-inflammatory mediators according to any one of preceding items.
17. The polypeptide for use as a medicament according to any one of preceding items wherein the polypeptide is a fragment of the Hpb glutamate dehydrogenase polypeptide, preferably said fragment having at least 20% or more (e.g., at least 30%, at least 40%, at least 50%, at least 60%, at least 70%, at least 80%, at least 90%, or 100%) of the polypeptide sequence of the Hpb glutamate dehydrogenase polypeptide, said glutamate dehydrogenase polypeptide having UniProtKB Accession Number: A0A183FP08 (e.g., SEQ ID NO: 1); preferably said polypeptide is capable of inducing of anti-inflammatory mediators according to any one of preceding items.
18. The polypeptide for use as a medicament according to any one of preceding items, wherein said polypeptide is selected from the group consisting of:
   i) Hpb glutamate dehydrogenase polypeptide, said glutamate dehydrogenase polypeptide having UniProtKB Accession Number: A0A183FP08;
   ii) Hpb ferritin polypeptide, said ferritin polypeptide having UniProtKB Accession Number A0A183FLG6 or A0A183FDM1;
   iii) Hpb aspartate aminotransferase polypeptide, said aspartate aminotransferase polypeptide having UniProtKB Accession Number: A0A183F107;
   iv) Hpb tubulin alpha chain polypeptide, said tubulin alpha chain polypeptide having UniProtKB Accession Number: A0A183GTY4, A0A183F2N5, A0A183FGY7, A0A183FJ38 or A0A183G7U3;
   v) Hpb histone H2B polypeptide, said histone H2B polypeptide having UniProtKB Accession Number: A0A183F3C5, A0A183FWH9, A0A183GMUO or A0A183GQR4;
   vi) Hpb proteasome subunit;
   vii) Hpb TCP-I/cpn60 chaperonin family protein;
   viii) Hpb myosin domain (e.g. N-terminal SH3-like domain, head domain),
   ix) Hpb Vitamin B12 binding domain,
   x) Hpb Immunoglobulin I-set domain,
   xi) Hpb Peptidase M17/Leucin Aminopeptidase,
   xii) Hpb Glycosyl hydrolases family 2, sugar binding domain,
   xiii) Hpb A-macroglobulin complement component,
   xiv) Hpb Enolase, N-terminal domain,
   xv) Hpb ERAP1-like C-terminal domain,
   xvi) Hpb ribosomal L5P family C-terminus,
   xvii) Hpb Acetyl-CoA hydrolase/transferase N-terminal domain,
   xviii) Hpb Cys/Met metabolism PLP-dependent enzyme,
   xix) Hpb Fructose bisphosphate aldolase,
   xx) Hpb Aminopeptidase I zinc metalloprotease (M18);
   xxi) Hpb Cysteine-rich secretory protein family member;
   xxii) the polypeptide as in defined (i)-(xxii), wherein said polypeptide is a fragment of the Hpb polypeptide as defined in (i)-(xxii), preferably said fragment having at least 20% or more (e.g., at least 30%, at least 40%, at least 50%, at least 60%, at least 70%, at least 80%, at least 90%, or 100%) of the polypeptide sequence of the Hpb polypeptide as defined in (i)-(xxii).
19. A nucleic acid encoding the polypeptide according to any one of preceding items.
20. An expression vector comprising at least one of the nucleic acid molecules according to any one of preceding items.
21. An isolated host cell comprising a vector and/or nucleic acid according to any one of preceding items.
22. An isolated antigen presenting cell exposed to the larval preparation, polypeptide, nucleic acid, expression vector or isolated host cell according to any one of preceding items.
23. The antigen presenting cell according to any one of preceding items, wherein said antigen presenting cell is a dendritic cell (e.g., a myeloid dendritic cell (mDC) or a plasmacytoid dendritic cells (pDC)).
24. The antigen presenting cell according to any one of preceding items, wherein said antigen presenting cell is a macrophage (e.g., an adipose tissue macrophage, monocyte, Kupffer cell, sinus histiocyte, alveolar macrophage, tissue macrophage, Langerhans cell, microglia cell, Hofbauer cell, intraglomerular mesangial cell, osteoclast, epithelioid cell, red pulp macrophage or peritoneal macrophage).
25. The antigen presenting cell according to any one of preceding items, wherein said antigen presenting cell is a B-cell (or B lymphocytes, e.g., plasmablast, plasma cell, lymphoplasmacytoid cell, memory B cell, follicular (FO) B Cell (also known as a B-2 cell), marginal zone (MZ) B cell, B-1 cell, B-2 cell, regulatory B (“Breg”) cell).
26. A composition comprising the larval preparation of Hpb helminths, polypeptide, nucleic acid, expression vector, isolated host cell or antigen presenting cell according to any one of preceding items.
27. The composition according to any one of preceding items, wherein said composition is a pharmaceutical or diagnostic composition.
28. The pharmaceutical composition according to any one of preceding items further comprising a pharmaceutically acceptable carrier and/or an anti-inflammatory agent, preferably said anti-inflammatory agent is one or more of the following: glucocorticoid, leukotriene receptor antagonist (LTRA, e.g., Montelukast, Zafirlukast or Pranlukast), mepolizumab, omalizumab 5-lipoxygenase inhibitor (e.g., Zileuton), leukotriene C4 synthase (LTCa synthase) inhibitor and FLAP inhibitor (e.g., 5-lipoxygenase activating protein inhibitor).
29. A vaccine or adjuvant comprising one or more of the following: larval preparation of Hpb helminths, polypeptide, nucleic acid, expression vector, isolated host cell or antigen presenting cell according to any one of preceding items; optionally, further comprising: a pharmaceutically accepted excipient or carrier.
30. A kit comprising the larval preparation of Hpb helminths, polypeptide, nucleic acid, expression vector, isolated host cell, antigen presenting cell, vaccine or adjuvant according to any one of preceding items.
31. A method for production of a cell-free L3-larval preparation of Heligmosomoides polygyrus bakeri (Hpb) helminths, said method comprising:
   i) homogenizing L3-developmental stage larvae of Hpb helminths; preferably said homogenizing is a homogenizing of sedimented L3-developmental stage larvae of Hpb helminths;
   ii) removing non-homogenized Hpb larval cells and cell debris followed by collecting resulting cell- and cell-debris free larval preparation, preferably said removing is carried out by centrifugation, wherein said larval preparation is collected in the form of supernatant;
   iii) optionally, heat and/or acid treating said resulting larval preparation of (ii), preferably said heat treating is carried out at 60° C. or 90° C. for 24 hours and/or said acid treating is carried out with 1M HCl at 60° C. for 24 hours;
   iv) isolating a polypeptide extract from said resulting larval preparation of (ii), preferably said polypeptide extract consisting essentially of polypeptides with molecular weight of 3 or more kDa; further preferably said polypeptide extract consisting essentially of polypeptides including oligomeric and/or monomeric polypeptides with molecular weight of monomeric polypeptides in the range of about 3-70 kDa, most preferably said polypeptide extract consisting essentially of polypeptides including oligomeric and/or monomeric polypeptides with molecular weight of monomeric polypeptides in the range of about 9-60 kDa; further most preferably said isolating is carried out by size exclusion chromatography, wherein said polypeptide extract is isolated in the form of protein fraction/s consisting essentially of polypeptides including oligomeric and/or monomeric polypeptides with molecular weight of monomeric polypeptides in the range of about 9-60 kDa;
   v) optionally, testing said protein fractions of (iv) for a capacity to modulate the innate mammalian immune system and discarding protein fractions that are not able to modulate the innate mammalian immune system.
32. The method for production of the larval preparation of Hpb helminths according to any one of preceding items, wherein said larval preparation is a cell-free larval preparation of Hpb helminths according to any one of preceding items.
33. A cell-free L3-larval preparation of Heligmosomoides polygyrus bakeri (Hpb) helminths produced by a method for production of a larval preparation of Hpb helminths according to any one of preceding items.
34. A method for treatment, amelioration, prophylaxis or diagnostics of a disease in a subject, said method comprising:
   i) providing the larval preparation of Hpb helminths, polypeptide, nucleic acid, expression vector, isolated host cell, antigen presenting cell, vaccine, adjuvant or kit according to any one of preceding items to said subject (e.g., human);
   ii) administering said larval preparation of Hpb helminths, polypeptide, nucleic acid, expression vector, isolated host cell, antigen presenting cell, vaccine, adjuvant or kit according to any one of preceding items to said subject;
   wherein said disease is selected from the group consisting of: chronic respiratory disease, steroid-resistant airway inflammation, aspirin-exacerbated respiratory disease (AERD), nasal polyps, cystic fibrosis (CF), allergic rhino-conjunctivitis, atopic dermatitis, autoimmune disease, inflammatory disease, chronic inflammatory disease, rhinitis, diabetes; bronchitis, chronic bronchitis, mucopurulent chronic bronchitis, emphysema, MacLeod syndrome, panlobular emphysema, centrilobular emphysema, chronic obstructive pulmonary disease (COPD), chronic obstructive pulmonary disease with acute lower respiratory infection, chronic obstructive pulmonary disease with acute exacerbation, asthma, predominantly allergic asthma, atopic asthma, extrinsic allergic asthma, non-allergic asthma, idiosyncratic asthma, intrinsic nonallergic asthma, mixed asthma, asthmatic bronchitis, late-onset asthma, status asthmaticus, acute severe asthma, bronchiectasis, nasal polyps, cystic fibrosis (CF), allergic rhino-conjunctivitis, atopic dermatitis, autoimmune or inflammatory disease, allergy, intolerance to painkillers (e.g., aspirin), nasal polyposis.
35. A method of eliciting or modulating an immune response in a subject, said method comprising:
   i) providing the larval preparation of Hpb helminths, polypeptide, nucleic acid, expression vector, isolated host cell, antigen presenting cell, vaccine, adjuvant or kit according to any one of preceding items to said subject (e.g., human);
   ii) administering said larval preparation of Hpb helminths, polypeptide, nucleic acid, expression vector, isolated host cell, antigen presenting cell, vaccine, adjuvant or kit according to any one of preceding items to said subject.
36. The method according to any one of preceding items, wherein said administering is not systemic.
37. The method according to any one of preceding items, wherein said administering is topical, preferably said topical administration is one or more of the following: enepidermic administration, epidermic administration, insufflation, irrigation, douching, painting or swabbing.
38. The method according to any one of preceding items, wherein said method is an in vitro, ex vivo or in vivo method.
39. The larval preparation of Hpb helminths, polypeptide, nucleic acid, expression vector, isolated host cell, antigen presenting cell, vaccine, adjuvant or kit according to any one of preceding items for use as medicament.
40. The larval preparation of Hpb helminths, polypeptide, nucleic acid, expression vector, isolated host cell, antigen presenting cell, vaccine, adjuvant or kit according to any one of preceding items for use in one or more of the following methods:
   i) in a method for modulating the mammalian innate immune response;
   ii) in a method for predominantly modulating the mammalian innate immune response over the mammalian adaptive immune response;
   iii) in a method for targeting phagocytic cells of the mammalian immune system;
   iv) in a method for modifying the activation of macrophages and/or granulocytes of the mammalian immune system;
   v) in a method for modifying the activation of one or more of the leukotriene pathway of the mammalian immune system;
   vi) in a method for decreasing the number of eosinophils and/or inhibiting the migration of granulocytes into tissue of the mammalian immune system;
   vii) in a method for inhibiting tissue infiltration with neutrophils and/or eosinophils in the mammalian immune system;
   viii) in a method for binding an iron atom associated with the mammalian arachidonate 5-lipoxygenase (5-LOX) enzyme and/or iron atom associated with mammalian cyclooxygenase (COX) enzyme;
   ix) in a method for reducing the expression and/or inhibiting one or more of the following: chemotactic receptors; cysteinyl leukotriene receptor 1 (CYSLTR1), preferably said CYSLTR1 is expressed by eosinophils; leukotriene C4 synthase (LTCa synthase);
   x) in a method for suppressing production of leukotrienes;
   xi) in a method for inducing of anti-inflammatory mediators;
   xii) in a method for reducing granulocyte recruitment and/or activation;
   xiii) in a method for inhibiting of arachidonate 5-lipoxygenase (5-LOX) having EC 1.13.11.34 enzymatic activity;
   xiv) in a method for eliciting or modulating an immune response in a subject;
   xv) in a method for suppressing type 2 helper (T_{H}2) cells differentiation and/or survival;
   xvi) in a method for producing an adjuvant, preferably said adjuvant for an allergen-specific immunotherapy;
   xvii) in a method for treatment, amelioration, prophylaxis or diagnostics of a steroid-resistant disease.
   xviii) in a method for treatment, amelioration, prophylaxis or diagnostics of a disease selected from the group consisting of: chronic respiratory disease, steroid resistant airway inflammation, aspirin-exacerbated respiratory disease (AERD), nasal polyps, cystic fibrosis (CF), allergic rhino-conjunctivitis, atopic dermatitis, autoimmune disease, inflammatory disease, chronic inflammatory disease, rhinitis, diabetes; bronchitis, chronic bronchitis, mucopurulent chronic bronchitis, emphysema, MacLeod syndrome, panlobular emphysema, centrilobular emphysema, chronic obstructive pulmonary disease (COPD), chronic obstructive pulmonary disease with acute lower respiratory infection, chronic obstructive pulmonary disease with acute exacerbation, asthma, predominantly allergic asthma, atopic asthma, extrinsic allergic asthma, non-allergic asthma, idiosyncratic asthma, intrinsic nonallergic asthma, mixed asthma, asthmatic bronchitis, late-onset asthma, status asthmaticus, acute severe asthma, bronchiectasis, nasal polyps, cystic fibrosis (CF), allergic rhino-conjunctivitis, atopic dermatitis, autoimmune or inflammatory disease, allergy, intolerance to painkillers (e.g., aspirin), nasal polyposis;
   xix) in a method for monitoring development of a disease and/or assessing the efficacy of a therapy of a disease;
   xx) in a method for screening a candidate compound for activity against a disease;
   xxi) in a method for assessing eosinophils-associated effects in chronic respiratory disease, aspirin-exacerbated respiratory disease (AERD), nasal polyps, Cystic fibrosis (CF), allergic rhino-conjunctivitis, atopic dermatitis, autoimmune or inflammatory disease, intolerance to painkillers (e.g., aspirin);
   xxii) in a method according to any one of preceding items;
   xxiii) in a method according to (i)-(xxii), wherein said disease is selected from the group consisting of: chronic respiratory disease, steroid resistant airway inflammation, aspirin-exacerbated respiratory disease (AERD), nasal polyps, cystic fibrosis (CF), allergic rhino-conjunctivitis, atopic dermatitis, autoimmune and inflammatory disease, chronic inflammatory disease, rhinitis, diabetes; bronchitis, chronic bronchitis, mucopurulent chronic bronchitis, emphysema, MacLeod syndrome, panlobular emphysema, centrilobular emphysema, chronic obstructive pulmonary disease (COPD), chronic obstructive pulmonary disease with acute lower respiratory infection, chronic obstructive pulmonary disease with acute exacerbation, asthma, predominantly allergic asthma, atopic asthma, extrinsic allergic asthma, non-allergic asthma, idiosyncratic asthma, intrinsic nonallergic asthma, mixed asthma, asthmatic bronchitis, late-onset asthma, status asthmaticus, acute severe asthma, bronchiectasis, nasal polyps, cystic fibrosis (CF), allergic rhino-conjunctivitis, atopic dermatitis, autoimmune or inflammatory disease, steroid-resistant chronic respiratory disease or a steroid resistant airway inflammation, allergy, intolerance to painkillers (e.g., aspirin), nasal polyposis.
41. Use of the larval preparation of Hpb helminths, polypeptide, nucleic acid, expression vector, isolated host cell, antigen presenting cell, vaccine, adjuvant or kit according to any one of preceding items for one or more of the following:
   i) for modulating the mammalian innate immune response;
   ii) for predominantly modulating the mammalian innate immune response over the mammalian adaptive immune response;
   iii) for targeting phagocytic cells of the mammalian immune system;
   iv) for modifying the activation of macrophages and/or granulocytes of the mammalian immune system;
   v) for modifying the activation of leukotrienes of the mammalian immune system;
   vi) for decreasing the number of eosinophils and/or inhibiting the migration of granulocytes into tissue of the mammalian immune system;
   vii) for inhibiting tissue infiltration with neutrophils and/or eosinophils in the mammalian immune system;
   viii) for binding an iron atom associated with the mammalian arachidonate 5-lipoxygenase (5-LOX) enzyme and/or iron atom associated with mammalian cyclooxygenase (COX) enzyme;
   ix) for reducing the expression and/or inhibiting one or more of the following: chemotactic receptors; cysteinyl leukotriene receptor 1 (CYSLTR1), preferably said CYSLTR1 is expressed by eosinophils; leukotriene C4 synthase (LTC₄ synthase);
   x) for suppressing production of leukotrienes;
   xi) for inducing of anti-inflammatory mediators;
   xii) for reducing granulocyte recruitment and/or activation;
   xiii) for inhibiting of arachidonate 5-lipoxygenase (5-LOX) having EC 1.13.11.34 enzymatic activity;
   xiv) for eliciting or modulating an immune response in a subject;
   xv) for suppressing type 2 helper (T_{H}2) cells differentiation and/or survival;
   xvi) for producing an adjuvant, preferably said adjuvant for an allergen-specific immunotherapy;
   xvii) for treatment, amelioration, prophylaxis or diagnostics of a steroid-resistant disease;
   xviii) for treatment, amelioration, prophylaxis or diagnostics of a disease selected from the group consisting of: chronic respiratory disease, steroid resistant airway inflammation, aspirin-exacerbated respiratory disease (AERD), nasal polyps, cystic fibrosis (CF), allergic rhino-conjunctivitis, atopic dermatitis, autoimmune disease, inflammatory disease, chronic inflammatory disease, rhinitis, diabetes; bronchitis, chronic bronchitis, mucopurulent chronic bronchitis, emphysema, MacLeod syndrome, panlobular emphysema, centrilobular emphysema, chronic obstructive pulmonary disease (COPD), chronic obstructive pulmonary disease with acute lower respiratory infection, chronic obstructive pulmonary disease with acute exacerbation, asthma, predominantly allergic asthma, atopic asthma, extrinsic allergic asthma, non-allergic asthma, idiosyncratic asthma, intrinsic nonallergic asthma, mixed asthma, asthmatic bronchitis, late-onset asthma, status asthmaticus, acute severe asthma, bronchiectasis, nasal polyps, cystic fibrosis (CF), allergic rhino-conjunctivitis, atopic dermatitis, autoimmune or inflammatory disease, allergy, intolerance to painkillers (e.g., aspirin), nasal polyposis;
   xix) for monitoring development of a disease and/or assessing the efficacy of a therapy of a disease;
   xx) for screening a candidate compound for activity against a disease;
   xxi) for assessing eosinophils-associated effects in chronic respiratory disease, aspirin-exacerbated respiratory disease (AERD), nasal polyps, Cystic fibrosis (CF), allergic rhino-conjunctivitis, atopic dermatitis, autoimmune or inflammatory disease;
   xxii) use in a method according to any one of preceding items;
   xxiii) use in a method according to (i)-(xxii), wherein said disease is selected from the group consisting of: chronic respiratory disease, steroid resistant airway inflammation, aspirin-exacerbated respiratory disease (AERD), nasal polyps, cystic fibrosis (CF), allergic rhino-conjunctivitis, atopic dermatitis, autoimmune and inflammatory disease, chronic inflammatory disease, rhinitis, diabetes; bronchitis, chronic bronchitis, mucopurulent chronic bronchitis, emphysema, MacLeod syndrome, panlobular emphysema, centrilobular emphysema, chronic obstructive pulmonary disease (COPD), chronic obstructive pulmonary disease with acute lower respiratory infection, chronic obstructive pulmonary disease with acute exacerbation, asthma, predominantly allergic asthma, atopic asthma, extrinsic allergic asthma, non-allergic asthma, idiosyncratic asthma, intrinsic nonallergic asthma, mixed asthma, asthmatic bronchitis, late-onset asthma, status asthmaticus, acute severe asthma, bronchiectasis, nasal polyps, cystic fibrosis (CF), allergic rhino-conjunctivitis, atopic dermatitis, autoimmune or inflammatory disease, steroid-resistant chronic respiratory disease or a steroid resistant airway inflammation, allergy, intolerance to painkillers (e.g., aspirin), nasal polyposis.
42. The use according to any one of preceding items, wherein said use is an in vitro, ex vivo or in vivo use.

### EXAMPLES OF THE INVENTION

In order that the invention may be readily understood and put into practical effect, some aspects of the invention are described by way of the following non-limiting examples.

### Example 1

### Example 1.1: Preparation of Hpb Larval Homogenate

Infective stage-three larvae (L3) of the nematode H. polygyrus bakeri (Hpb) were obtained by previously published methods (Camberis et al. 2003) and washed twice in sterile PBS supplemented with antibiotics (Penicillin, Streptomycin). Sedimented larvae were homogenized in a Precellys homogenizer. Remaining debris were removed by centrifugation and aliquots of the resulting supernatants were stored at −80° C. until use.

In some experiments, Hpb homogenate was subjected to heat treatment (e.g., 60° C. or 90° C. for 24 hours to denature proteins or to acid treatment (e.g., 1 M HCl, 60° C., 24 hrs.) to destroy carbohydrate structures.

### Example 1.2: Fractionation of Hpb Extract

Hpb protein extract was fractionated by size exclusion chromatography (e.g., gel filtration via Superdex 75 column) and the resulting protein fractions were tested for their immunomodulatory activity in cellular assays (see below).

### Example 1.3: MS Identification of Candidate Immunomodulatory Proteins in Hpb Extract

Active (and inactive) protein fractions of Hpb extract were subjected to mass spectrometry (MS) analysis. Candidate immunomodulatory proteins were identified by comparing the protein composition of active and inactive fractions. MS score was used for selection of candidate proteins. The two major immunomodulatory candidates identified are:

Hpb Ferritin and Hpb Glutamate dehydrogenase.

Further candidates included:

Hpb Aspartate transaminase/Aspartate aminotransferase,

Tubulin alpha chain,

Histone H2B,

Proteasome subunits and several uncharacterized proteins of Hpb, including

TCP-I/cpn60 chaperonin family,

Myosin domains (e.g. N-terminal SH3-like domain, head domain),

Vitamin B12 binding domain,

Immunoglobulin I-set domain,
Peptidase M17/Leucin Aminopeptidase,
Glycosyl hydrolases family 2, sugar binding domain,
A-macroglobulin complement component,
Enolase, N-terminal domain,
ERAP1-like C-terminal domain,
ribosomal L5P family C-terminus,
Acetyl-CoA hydrolase/transferase N-terminal domain,
Cys/Met metabolism PLP-dependent enzyme,
Fructose bisphosphate aldolase (FBPA),
Aminopeptidase I zinc metalloprotease (M18) and
Cysteine-rich secretory protein family members.
Starting with the candidates with the highest MS score candidate proteins could be recombinantly produced and tested individually and in different combinations in the following cellular assays (see below).

### Example 1.4: Recombinant Production and Purification of Candidate Proteins

Hpb Ferritin and Hpb Glutamate dehydrogenase were cloned into suitable expression vectors and produced recombinantly in E. coli or mammalian expression systems (e.g. HEK cells). Recombinant proteins were purified by size exclusion chromatography and tested individually or in combination for their activity in cellular assays (see below).

### Example 1.5: Cellular Assays

Human polymorphonuclear leukocytes (PMN) or peripheral blood mononuclear cells (PBMC) were isolated from the peripheral blood from healthy controls or patients suffering from AERD using density gradient centrifugation. For some experiments, cells were further separated into monocytes (CD14⁺), neutrophils (CD16⁺) or eosinophils. CD14⁺ monocytes were differentiated into macrophages by culture for 6-8 days in the presence of GM-CSF and TGFbl. In most experiments, cells were treated with Hpb proteins at a concentration of 10 μg protein/ml.

Cellular Assay 1: Mediator Production

Mediator analysis in cell supernatants (+/−Hpb proteins) was performed by immunoassay for individual mediators (e.g. LTs, PGE2 or IL-10) or LC-MS/MS or Multiplex cytokine analysis for overall mediator profiles.

Cellular Assay 2: Chemotaxis

Granulocyte recruitment (induced by chemokines or nasal polyp secretions) was assessed with or without pre-treatment with Hpb proteins by using trans-well assays. Migrated granulocytes were enumerated microscopically and by flow cytometry.

Topical Administration of Hpb Proteins in an In Vivo Allergy Model

Mice were treated with the total Hpb homogenate (protein mixture) intranasally during sensitization and challenges with house dust mite allergens. Infiltration of inflammatory cells (including eosinophils) into the airways was analyzed by flow cytometric analysis and cytospins of bronchoalveolar lavage fluid. Airway inflammation was assessed by histology.

Results of Cellular Assays:

Hpb proteins (e.g., total somatic homogenate of Hpb L3 larvae) broadly modulate mediator profiles of human myeloid cells. In order to mimic a clinically relevant inflammatory setting, human granulocytes (PMN) were treated with GM-CSF (100 ng/ml), a pro-inflammatory cytokine and granulocyte survival factor, which is particularly increased in nasal polyps (Stevens et al. 2015) and associated with steroid resistance (I to et al. 2008, da Silva Antunes et al. 2015). After 16h of culture, GM-CSF treated mixed human PMN showed 50-90% viability and pronounced LT production. Treatment with Hpb proteins (e.g., total somatic homogenate of Hpb L3 larvae) resulted in a two- to four-fold reduction in LTs (p=0.004) (n=9).

Mixed PMN contain 80-97% neutrophils and thus produce mainly LTB₄ (Leukotriene B4). However, in inflamed airway tissue, eosinophils often represent the dominant granulocyte population. Thus, purified human eosinophils (purity 95-99%) were treated with Hpb proteins (e.g., total somatic homogenate of Hpb L3 larvae). Despite considerable donor variation in the production of LTs, GM-CSF treated eosinophils from all donors (n=8) showed a dramatic (50-4000 fold) reduction in the production of LTs (p=0.007) after treatment with said Hpb proteins.

Although much less dramatic, there was also a tendency for reduced LTB4 production by eosinophils (p=0.07, 7 out of 9 donors).

In order to study the effect of Hpb proteins on human macrophages, human monocyte derived macrophages (MDM) were stimulated with A23187 after 16 hrs. treatment with helminth proteins (e.g., total somatic homogenate of Hpb L3 larvae). Human macrophages responded to treatment with a tendency of reduced production of 5-lipoxygenase metabolites (including LTs), whilst the production of anti-inflammatory PGE₂ was markedly (450-fold) induced (p=0.002, n=10).

These data were also confirmed by qPCR (quantitative polymerase chain reaction) and western blot analysis, which showed that the expression of LT-producing enzymes is suppressed, whilst the expression of PG-producing enzymes (e.g. cyclooxygenase-2, microsomal prostaglandin E2 synthase 1) was induced.

In addition, Hpb treatment of macrophages (e.g., with total somatic homogenate of Hpb L3 larvae) resulted in the induction (approximately 50-fold) of anti-inflammatory IL-10 (p<0.0001, n=23).

Hpb proteins also reduced markers of granulocyte activation and chemotaxis. Flow cytometric analysis showed that treatment with Hpb proteins (e.g., total somatic homogenate of Hpb L3 larvae) (10 μ/ml, 16h) reduced the surface levels of the eotaxin receptor CCR3 (C—C chemokine receptor type 3) on eosinophils from all donors by 2- to 6-fold (n=9; n=5 controls, n=4 patient samples, p=0.003).

Also, the levels of the prostaglandin D2 receptor CRTH2, a receptor implicated in airway inflammation (Nantel et al. 2004), were significantly reduced on eosinophils after treatment (p=0.007).

Treatment with Hpb proteins (e.g., total somatic homogenate of Hpb L3 larvae) also reduced granulocyte chemotaxis in response to nasal polyp secretions to baseline levels (e.g., 80% reduction, p=0.03) for cells derived from AERD patients (n=6) and healthy controls (n=3). Of note, fluticasone propionate (1 μM) failed to reduce chemotactic responses and the cysLT1R (selective cysteinyl leukotriene receptor 1) antagonist Montelukast (10 μM) only reduced chemotaxis by 10% (p=0.03). Thus, regarding an important anti-inflammatory effect, Hpb proteins were superior to standard treatments of chronic airway inflammation.

The cell viability after treatment with Hpb extract (e.g., total somatic homogenate of Hpb L3 larvae) and Montelukast was moderately reduced (from 88% to 79% (p=0.01) or 81% (p=0.007), respectively), whilst fluticasone propionate had no significant effect on the viability of granulocytes. All granulocyte cultures were performed in the presence of 100 ng/ml GM-CSF to suppress apoptosis and simulate the inflammatory environment of nasal polyps or asthmatic lung tissue. The tendency of Hpb extract to reduce granulocyte survival might also add to its therapeutic effect as granulocyte removal is a desired outcome of anti-inflammatory drugs.

A mixture of Hpb proteins reduces airway inflammation in vivo. To confirm a potential efficacy of Hpb proteins (e.g., total somatic homogenate of Hpb L3 larvae) during airway inflammation in vivo, mice were treated with the total somatic homogenate of Hpb L3 larvae during allergic airway inflammation induced by house dust mite (HDM). Intranasal treatment with Hpb homogenate (containing Hpb proteins) reduced the HDM-triggered airway eosinophilia, resulting in an approximately 4-fold reduction in airway eosinophil numbers.

It was also shown that Hpb proteins (e.g., total somatic homogenate of Hpb L3 larvae) could suppress airway inflammation in mice in vivo, when applied topically, which represents an advantage compared to systemic treatment with current immunomodulatory proteins such as monoclonal antibodies (e.g., mepolizumab, omalizumab).

Conclusions:

In the course of the present invention proteins were identified and isolated from Hpb that are capable of broadly modulating inflammatory responses, by (i) suppressing the production of LTs, (ii) inducing the production of anti-inflammatory mediators (prostaglandin E2, IL-10) and (iii) reducing granulocyte recruitment and activation. Thus, the identified proteins target several key mechanisms of chronic airway inflammation at the same time. None of the currently available anti-inflammatory drugs (e.g. glucocorticosteroids, LT receptor antagonist (LTRA) (e.g., Montelukast, mepolizumab) shows a similar profile of activities. It was also shown that Hpb proteins could suppress airway inflammation in mice in vivo, when applied topically, which represents an advantage compared to systemic treatment with current immunomodulatory proteins such as monoclonal antibodies (e.g., mepolizumab). Due to the capacity to induce PGE2 and IL-10, Hpb proteins could potentially be used to suppress TH2 differentiation and are thus interesting candidates for improving the efficacy of allergen specific immunotherapy.

### Example 2

### Example 2.1: Hpb L3 Larval Extract has Immuneregulatory Effects, which are Distinct from Commonly Used Glucocorticosteroids

For the treatment of complex type 2 inflammatory diseases such as allergy, asthma and nasal polyps, regulation of multiple pathways is superior to targeting single mechanisms. Thus, glucocorticosteroids (GCs), which regulate a broad array of inflammatory pathways are widely used in the treatment of these diseases and represent the current first-line therapy for most patients. However, GCs lack efficacy in many patients, particularly in those suffering from severe leukotriene-driven type 2 inflammation (e.g. Aspirin exacerbated respiratory disease (AERD)).

Thus, the inventors compared the immune regulatory effects of Hpb L3 larval extract (HpbE) to those of glucocorticosteroids (dexamethasone and fluticasone propionate) with a focus on eicosanoid pathways and the anti-inflammatory cytokine IL-10. As shown in FIG. 1, HpbE suppressed type 2-inducing pathways such as the enzymatic machinery for cysLT and PGD₂ generation (ALOX5, LTC4S, PTGDS), whilst inducing anti-inflammatory mediator pathways (PGE₂ and IL-10) when administered to human monocyte derived macrophages (MDM). In contrast, GCs even tended to enhance LTC4S expression and to suppress PGE₂ synthesis and they did not enhance IL-10 production by macrophages. In line with the ELISA data for PGE₂, HpbE strongly induced the overall generation of COX metabolites, but reduced 5-LOX metabolites (5-HETE, 5-oxo-ETE and leukotrienes) (measured by LC-MS/MS), two effects which were not observed for GC treatment of human macrophages (FIG. 1C).

The inventors also assessed the modulation of eicosanoids in human granulocytes (PMN) and observed that HpbE and FP could both reduce pro-inflammatory arachidonic acid (AA) and linoleic acid (LA) metabolites (1) in these cells (FIG. 1D). In contrast, the generation of COX metabolites by PMN was relatively low and not affected by either HpbE or GC treatment. Together, this suggested that HpbE is superior to GCs in inducing a regulatory mediator profile that could counteract type 2 inflammation.

### Example 2.2: HpbE, but not Fluticasone Propionate Induces a Regulatory and Tissue-Reparative Eicosanoid Profile in Macrophages from AERD Patients

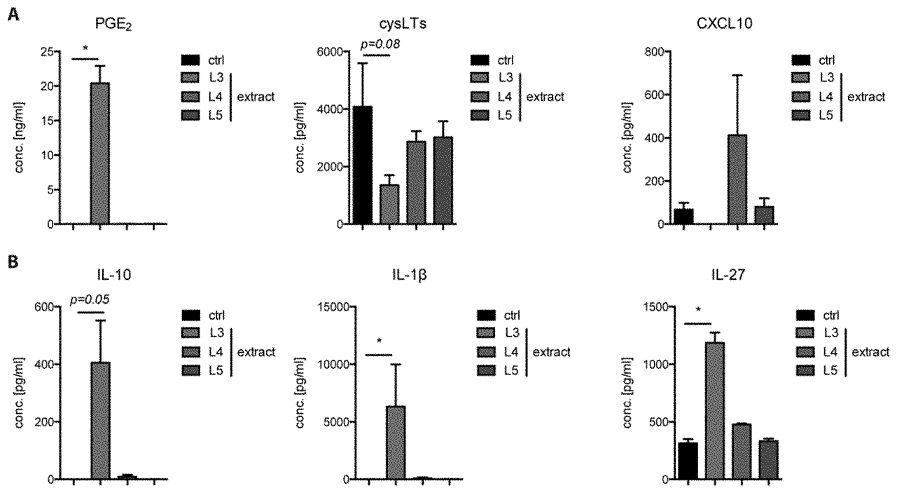

To validate the efficacy of HpbE in a relevant therapeutic indication, the inventors generated MDM from AERD patients and studied the effects of HpbE and FP on the mediator output. As shown in FIG. 2, HpbE efficiently increased COX metabolites and 15-LOX metabolites, while decreasing 5-LOX metabolites in MDM form AERD patients.

Importantly, COX and 15-LOX metabolites, have regulatory/tissue reparative functions, whilst 5-LOX metabolites are pro-inflammatory and drive tissue damage (for a recent review see Esser-von Bieren et al. (2019), Immunology & Cell Biology, 97(3):279-288). In contrast, FP had only minor effects on the eicosanoid output in MDM from healthy individuals (weak induction of PGE₂), whilst no effects on the eicosanoid output of MDM from AERD patients could be observed. Thus, HpbE not only reduced the migration of AERD granulocytes, but also suppressed leukotriene production by AERD macrophages. As AERD represents a severe type 2 inflammatory disease, which is characterized by (partial) resistance to GCs, HpbE-based therapeutics represent an attractive alternative or add-on treatment for AERD.

### Example 2.3: L4 and L5 Stage Extracts of H. Polygyrus Bakeri Fail to Induce Type 2-Suppressive Mediators

To test whether the observed immuneregulatory effects were unique to L3 as compared to L4 or L5 extracts, the inventors additionally homogenized L4 and L5 stages of Hpb and administered the resulting extracts in their macrophage assays. As shown in FIG. 3A, L4 as well as L5 extracts of Hpb failed to induce PGE₂ and only showed minor suppressive effects on cysLTs as compared to L3 stage extract (HpbE).

In contrast, L4 extract induced CXCL10, a chemokine associated with severe, corticosteroid-resistant asthma, an effect, which was not observed for L3 or L5 extracts (FIG. 3A) (Gauthier et al. (2017), JCI Insight, 2 (13):e94580). In addition, in contrast to L3 extract, L4 and L5 extracts did not induce the release of regulatory and type-2 suppressive cytokines (IL-1β, IL-10 and IL-27) (FIG. 3B) (Nguyen et al. (2017), JCI Insight, 4(2):e123216).

Together this suggested that L3 extract has immuneregulatory properties that are distinct from both L4 and L5 stage extracts, thus rendering L3 extract (HpbE) a unique source of type 2-suppressive factors.

### Example 2.4: Glutamate Dehydrogenase is a Major Immunoregulatory Protein in HpbE

To characterize the molecules responsible for the immunoregulatory effects of HpbE, the inventors analyzed prostanoid and cytokine production by MDM as well as chemotaxis of granulocytes after treatment with heat-inactivated HpbE. Heat-inactivation of HpbE attenuated the induction of prostanoids, IL-10 and IL-1β in MDM as well as the HpbE-driven suppression of granulocyte recruitment (FIG. 4A). In addition, the induction of IL-10 by HpbE was abrogated if the extract was pre-treated with proteinase K (FIG. 4B). This suggested that mediator reprogramming by HpbE was largely dependent on heat-labile and proteinase K digestible molecules, most likely proteins.

In order to identify immuneregulatory proteins present in HpbE, the inventors fractionated the extract by size exclusion chromatography and identified active fractions (8-11) based on the capacity to induce the COX metabolite TXB₂ as well as IL-10 (FIGS. 4C and 4D). The inventors then identified proteins present in active and non-active fractions by mass spectrometry, thus highlighting Hpb glutamate dehydrogenase (GDH) as a major immuneregulatory candidate, which was uniquely present in active fractions of HpbE (summarized in FIG. 4E). In addition, an inhibitor of GDH (Bithionol), which is also used as an anti-helminthic, reduced the HpbE-triggered induction of PGE₂ and IL-10 (FIG. 4F).

As a new tool for studying the uptake, localization and function of Hpb GDH in vivo and in target cells in vitro, we generated monoclonal antibodies (mABs) specific for Hpb GDH (i.e. not cross-reactive with mammalian (human/mouse) GDH) (FIG. 5). Clone 4F8 was selected as the best candidate for further sub cloning and for testing in neutralization experiments. Indeed, addition of 4F8 to macrophage cultures during HpbE treatment resulted in a dose-dependent reduction of HpbE-induced IL-10 and PGE₂ production (FIG. 4H).

The inventors also developed a strategy for the overexpression and purification of recombinant Hpb GDH (containing a His-Tag) in E. coli. Expression at low temperatures (16° C.) was used to obtain soluble Hpb GDH for further purification and testing in macrophage assays. Recombinant Hpb GDH obtained by the current protocol was still immunologically active as it could induce PGE₂ and IL-10 production by human macrophages (FIG. 4I). This effect could be attenuated by addition of the 4F8 mAb directed against Hpb GDH (FIG. 4I). Moreover, recombinant Hpb GDH was able to reduce the generation of pro-inflammatory cysLTs by human macrophages and the anti-Hpb GDH antibody (4F8) restored cysLT levels in Hpb GDH-treated cells to a large extent (FIG. 4I).

Taken together, the inventors identified the metabolic enzyme Hpb GDH as a major protein component of HpbE that is involved in the immuneregulatory effects of the Hpb L3 larval extract (HpbE).

### Materials and Methods

### Macrophage Assays

Human monocyte derived macrophages (MDM) were isolated, cultured and stimulated as described herein, e.g. in Example 3.

### Mediator Analysis

Eicosanoids or cytokines were quantified by LC-MS/MS or immunoassays as described herein, e.g. in Example 3.

### Fractionation and Mass Spectrometry Analysis of Hpb Larval Extract

Soluble protein fractions were separated by gel filtration chromatography (SEC) on a Superdex 75 10/300 GL column with the ÄKTA pure system (GE Health Care Life Science). 300 μl of Hpb extract was loaded onto the column and eluted isocratically with PBS (pH=8), flow rate 0.8 ml/min. Fractions of 0.5 ml were collected starting when protein presence was detected at λ=280 nm.

Fractions from the SEC were prepared for liquid chromatography-mass spectrometry analysis, as described previously (Bepperling et al. (2012), PNAS 109:20407-20412, Mymrikov (2017), J Biol Chem, 292:672-684). Proteins in the samples were reduced, alkylated and digested overnight with trypsin. Peptides were extracted in five steps by adding sequentially 200 μl of buffer A (0.1% formic acid in water), acetonitrile (ACN), buffer A, ACN, ACN respectively. After each step samples were treated for 15 min by sonication. After steps 2, 4 and 5, the supernatant was removed from the gel slices and collected for further processing. The collected supernatants were pooled, evaporated to dryness in a speed vac (DNA 120, ThermoFisher Scientific) and stored at −80° C. For the MS measurements the samples were dissolved by adding 24 μl of buffer A and sonicated for 15 min. The samples were then filtered through a 0.22-μm centrifuge filter (Merck Millipore). Peptides were loaded onto an Acclaim PepMap RSLC C18 trap column (Trap Column, NanoViper, 75 μm×20 mm, C18, 3 μm, 100 A, ThermoFisher Scientific) with a flow rate of 5 μL/min and separated on a PepMap RSLC C18 column (75 μm×500 mm, C18, 2 μm, 100 A, ThermoFisher Scientific) at a flow rate of 0.3 μL/min. A double linear gradient from 5% (vol/vol) to 28% (vol/vol) buffer B (acetonitrile with 0.1% formic acid) in 30 min and from 28% (vol/vol) to 35 (vol/vol) buffer B in 5 min eluted the peptides to an Orbitrap QExactive plus mass spectrometer (ThermoFisher Scientific). Full scans and five dependent collision-induced dissociation MS2 scans were recorded in each cycle.

The mass spectrometry data derived from the SEC fractions were searched against the Swiss-Prot Heligmosomoides polygyrus bakeri Database downloaded from UniProt (24.01.2017 edition) using the Sequest HT Algorithm implemented into the “Proteome Discoverer 1.4” software (ThermoFisher Scientific). The search was limited to tryptic peptides containing a maximum of two missed cleavage sites and a peptide tolerance of 10 ppm for precursors and 0.04 Da for fragment masses. Proteins were identified with two distinct peptides with a target false discovery rate for peptides below 1% according to the decoy search. Proteins detected in the negative control samples were subtracted from the respective hit-lists. For further evaluation two independent datasets resulting from SEC separations of biological replicates were combined. Only hits that were observed in both datasets were taken into account.

### Recombinant Expression and Purification of Hpb GDH

E. coli BL21 transformed with pET21a HpbGDH, was grown in 50 ml Luria Broth (LB) containing ampicillin (100 μg/ml) for 16 h at 37° C. 1L expression culture was inoculated with 1:100 pre-culture and incubated at 37° C., 180 rpm until the OD₆₀₀ reached 0.6. Isopropyl-β-D-thiogalactopyranosid (IPTG) was added to a final concentration of 1 mM and the protein expression was done at 16° C., 150 rpm for 16 h. Bacteria were harvested by centrifugation (45 min, 4100×g, 20° C.). The bacterial pellet was washed in PBS and resuspended in 50 mM NaH₂PO₄ (pH 8.0), 300 mM NaCl, 10 mM imidazole. Subsequently the resuspended cells were treated with DNAse I and the soluble fraction was obtained by sonication followed by centrifugation (20,000 g, 45 min, 4° C.). The supernatant was applied to a HisTrap HP column (GE Healthcare) in 50 mM NaH₂PO₄ (pH 8.0), 300 mM NaCl, 10 mM imidazole. Elution was performed in 50 mM NaH₂PO₄ (pH 8.0), 300 mM NaCl, 250 mM imidazole. The protein containing eluate fractions were applied to a Superose® 6 Increase 10/300 GL column (GE Healthcare) equilibrated in 50 mM NaH₂PO₄ (pH 8.0), 300 mM NaCl. After gel filtration, the protein containing fractions (F-16-F18) were reconcentrated and used for macrophage assays. The protein concentration was determined by NanoPhotometer N60 (Implen).

### Generation of Monoclonal Antibodies Against Hpb GDH

Rats were immunized against two different peptides specifically found in GDH of Hpb, but not mammalian GDH (peptides A and B are specified in FIG. 4). The subsequent steps (fusion, hybridoma screening and sub cloning) were carried out according to standard procedures of the monoclonal antibody core facility at the Helmholtz Center Munich (https://www.helmholtz-muenchen.de/mab/how-we-work/index.html). Westernblot analysis was performed according to previously published protocols (Dietz et al. (2016), J. Allergy Clin. Immunol, 139(4):1343-1354.e6).

Recovery and Homogenization of L4 and L5 Stages from Hpb Infected Mice

Mice were infected with 200 L3 of Hpb as described previously (Esser-von Bieren et al. (2013), PLoS Pathog. 9:e1003771) and L4 or L5 stages of Hpb were recovered from the intestine on day 6 or 10, respectively. Recovered L4 or L5 were homogenized as described for L3. The resulting extracts had a protein concentrations that were similar to L3 extract (range: 500-1000 μg/ml).

### Example 3

### Example 3.1: Helminth Larvae Trigger Local Remodeling of the Arachidonic Acid Metabolism

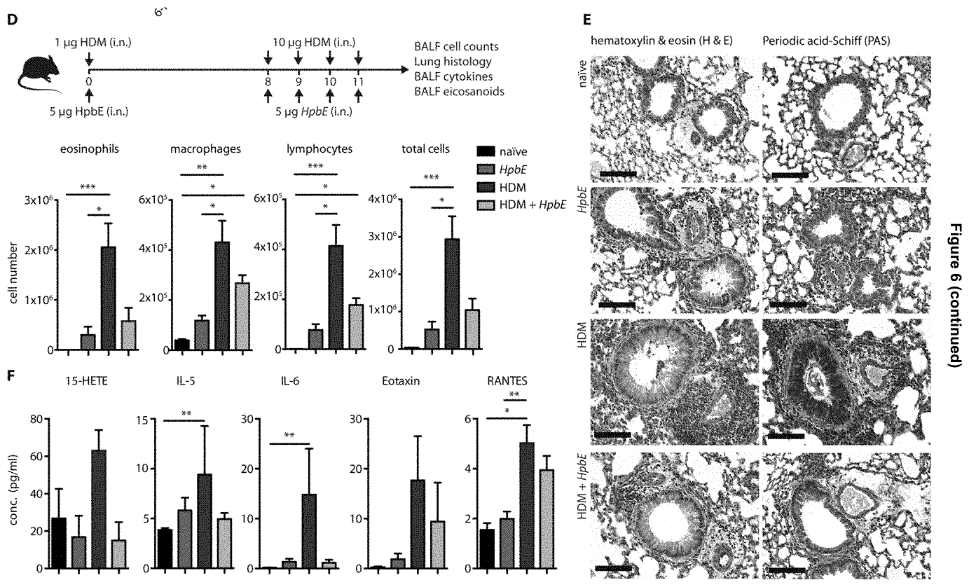

Type 2 immune responses in allergy and helminth infection are driven by pro-inflammatory changes in AA (arachidonic acid)-metabolic pathways. However, given that helminth parasites can negatively regulate type 2 immunity, the inventors sought to study whether helminths could trigger anti-inflammatory remodeling of the host AA metabolism. Thus, the inventors quantified AA metabolites in intestinal culture supernatants and peritoneal lavage of mice during early primary infection with Heligmosomoides polygyrus bakeri (Hpb) by liquid chromatography tandem mass spectrometry (LC-MS/MS). At this time point (day 7), Hpb larvae have invaded the intestinal wall and reside within the tissue. In general, the formation of AA metabolites in the intestine and peritoneal cavity was increased by Hpb infection (FIGS. 6A and B). High levels of prostanoids (PGE2, TXB2, 6-keto PGF1a and PGF2a) and 12/15-lipoxygenase (LOX) metabolites (12- and 15-hydroxyeicosatetraenoic acid (HETE)) were detected in samples from Hpb-infected mice, with levels in intestinal culture supernatants greatly exceeding those in peritoneal lavage (FIGS. 6A and 6B). In contrast, 5-LOX metabolites (5-HETE and leukotrienes (LTs)) were close to or below the lower limit of quantification (FIGS. 6A and B). In line with the abundant production of prostanoids, cyclooxygenase 2 (COX-2) and its positive regulator hypoxia inducible factor-1 alpha (HIF-1a), were abundant in the surrounding of Hpb larvae and in cells adjacent to larvae (FIG. 6C, top). In keeping with the absence of LTs, 5-LOX protein was absent from the surrounding of Hpb larvae in infected mice, whilst 5-LOX expressing cells were present in intestinal tissue of naïve mice (FIG. 6C, bottom). Thus, Hpb larvae triggered fundamental changes in the local AA metabolism.

### Example 3.2: Treatment with HpbE Suppresses Allergic Airway Inflammation In Vivo

As AA metabolites are critical regulators of the type 2 immune response to house dust mite (HDM), we tested how treatment with homogenized Hpb larvae (Hpb larval extract, “HpbE”) would affect HDM-induced allergic airway inflammation in vivo (FIG. 6D, top). Local (intranasal, i.n.) administration of HpbE reduced hallmarks of type 2 inflammation, including airway eosinophilia and mucus production (FIGS. 1D and E). Consistent with increased eosinophil numbers, 15-HETE, a major AA metabolite of eosinophils was increased in bronchoalveolar lavage fluid (BALF) of HDM-sensitized mice and treatment with HpbE tended to decrease 15-HETE levels as well as pro-inflammatory cytokines and chemokines (IL-5, IL-6, Eotaxin, RANTES) (FIG. 6F). Thus, local administration of HpbE could suppress the inflammatory response to HDM in the airways.

### Example 3.3: Modulation of Type 2 Inflammation by HpbE-Conditioned Macrophages Depends on COX-2 Metabolites

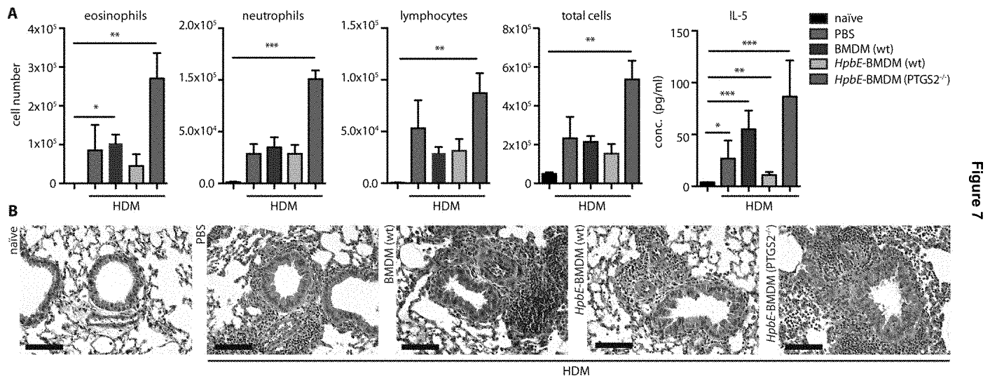

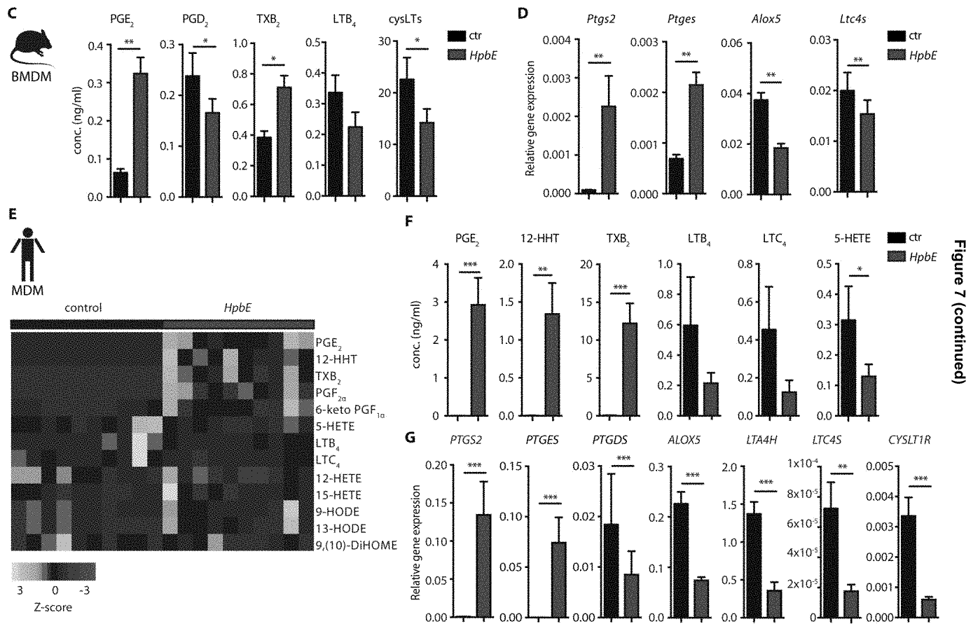

Macrophages are key producers of AA metabolites in the airways and monocytes/macrophages are recruited from the bone marrow and drive allergic airway inflammation in response to HDM. The inventors therefore assessed whether HpbE-treated macrophages could modify HDM-induced airway inflammation and if COX-2 contributed to this modulation by intranasal transferring bone marrow derived macrophages (BMDM) from wildtype or COX-2 deficient mice (PTGS2−/−). Mice that received untreated BMDM during experimental HDM allergy showed increased airway eosinophilia and inflammation as compared to control mice (FIGS. 7A and B). This pro-inflammatory effect was lost, when mice received wildtype BMDM that had been treated with HpbE (FIGS. 7A and B). In contrast, transfer of HpbE-treated PTGS2−/− BMDM resulted in exaggerated granulocyte recruitment and increased airway inflammation during HDM allergy (FIGS. 7A and B). This suggested that HpbE induces a COX-2 expressing regulatory macrophage phenotype, which is able to control granulocyte recruitment and type 2 inflammation.

### Example 3.4: HpbE Induces a Type 2-Suppressive Eicosanoid Profile in Murine and Human Macrophages

To characterize the eicosanoid profile of HpbE-induced type 2-suppressive macrophages, the inventors quantified key mediators of type 2 inflammation by LC-MS/MS. Consistent with the anti-inflammatory potential of HpbE-conditioned BMDM we observed a shift from type 2-inducing metabolites (PGD2, LTs) to regulatory metabolites (PGE2) after treatment with HpbE (FIG. 7C). This was likely a result of transcriptional changes in AA-metabolizing enzymes as HpbE induced COX-2 (gene: Ptgs2) and microsomal prostaglandin E synthase (mPGES-1, gene: Ptges), whilst suppressing 5-LOX (Alox5) and Ltc4s (leukotriene C4 synthase) gene expression (FIG. 7D). Thus, the eicosanoid profile of HpbE-conditioned BMDM resembled local AA metabolism changes during Hpb infection (FIGS. 6A and B).

To investigate whether the type 2-suppressive effects of HpbE could be translated to human macrophages, the inventors treated human monocyte derived macrophages (MDM) with HpbE and assessed their lipid mediator profile. Using an LC-MS/MS eicosanoid screen (including 200 different eicosanoids and PUFAs), we confirmed that HpbE treatment resulted in fundamental changes in AA metabolites, whilst LA metabolites (9-HODE, 13-HODE, 9, (10)-DiHOME) remained largely unaffected (FIG. 7E). As observed during Hpb infection and in HpbE-treated murine macrophages, COX-metabolites such as PGE2, TXB2 and 12-hydroxyheptadecatrenoic acid (12-HHT) were increased by HpbE (FIGS. 7E and F). In contrast, HpbE reduced the production of 5-LOX metabolites (5-HETE, LTB4 and LTC4) (FIGS. 7E and F), thus inducing a potentially anti-inflammatory eicosanoid signature.

In line with HpbE-induced transcriptional changes in mouse BMDM, human macrophages responded to HpbE by inducing the expression of enzymes involved in the biosynthesis of PGE2: PTGS2 (COX-2) and PTGES (mPGES-1) (FIG. 7G). In contrast, HpbE reduced the expression of PTGDS (prostaglandin D2 synthase) as well as of LT biosynthetic enzymes: ALOX5, LTA4H (leukotriene A4 hydrolase) and LTC4S and the high affinity receptor for cysLTs (Cysteinyl Leukotriene Receptor-1, CYSLTR1) (FIG. 7G). Taken together, HpbE triggered a switch from type 2-inducing to type 2-suppressive eicosanoid pathways in macrophages from both mice and humans.

### Example 3.5: HpbE Induces Type 2-Suppressive Cytokines and Prevents M2 Polarization

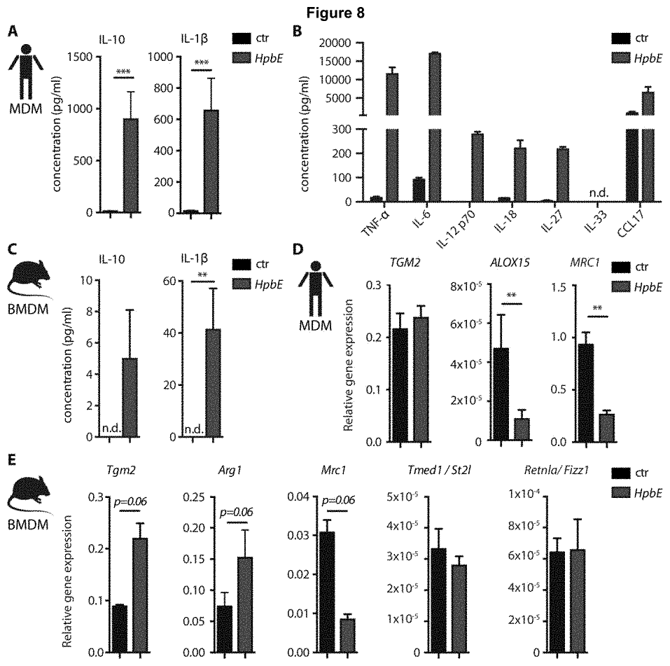

To investigate whether treatment with HpbE also modified cytokine profiles and the polarization of macrophages, the inventors quantified cytokines implicated in macrophage polarization and the regulation of type 2 inflammation. Treatment of human MDM with HpbE resulted in the induction of IL-10, IL-1β, IL-12, IL-18, IL-27 and TNF-α, all known to modulate M2 polarization and type 2 immune responses (FIGS. 8A and B). However, HpbE hardly affected the production of mediators of type 2 inflammation (IL-33 or CCL17) by macrophages (FIG. 8B). The HpbE-triggered induction of IL-10 and IL-1β also occurred in murine BMDM, albeit at 10-100-fold lower amplitude as compared to human MDM (FIG. 8C).

In addition, HpbE downregulated the expression of M2 markers (ALOX15 (15-Lipoxygenase, 15-LOX) and MRC1 (Mannose Receptor C-Type 1, MR/CD206)) in human MDM, suggesting that it could counteract M2 polarization (FIG. 8D). As human and mouse M2 macrophages are defined by distinct sets of markers, we also investigated the effect of HpbE on murine M2 polarization. In mouse BMDM, HpbE tended to induce Tgm2 and Arg1 expression but downregulated Mrc1 as in human MDM (FIG. 8E). Together, these data suggest that HpbE can broadly modulate the polarization and mediator output of macrophages to induce a regulatory, type 2-suppressive phenotype.

### Example 3.6: HpbE has a Unique Potential to Modulate the AA Metabolism

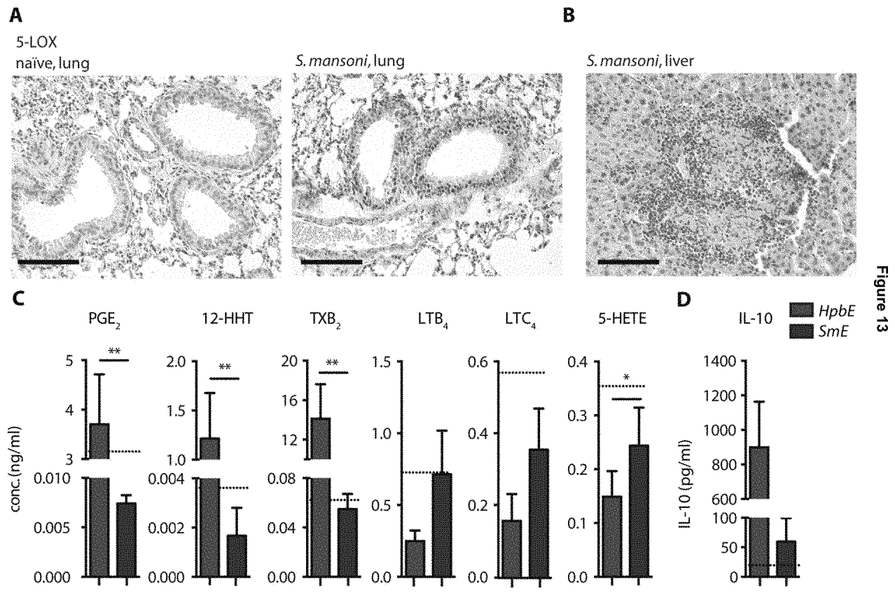

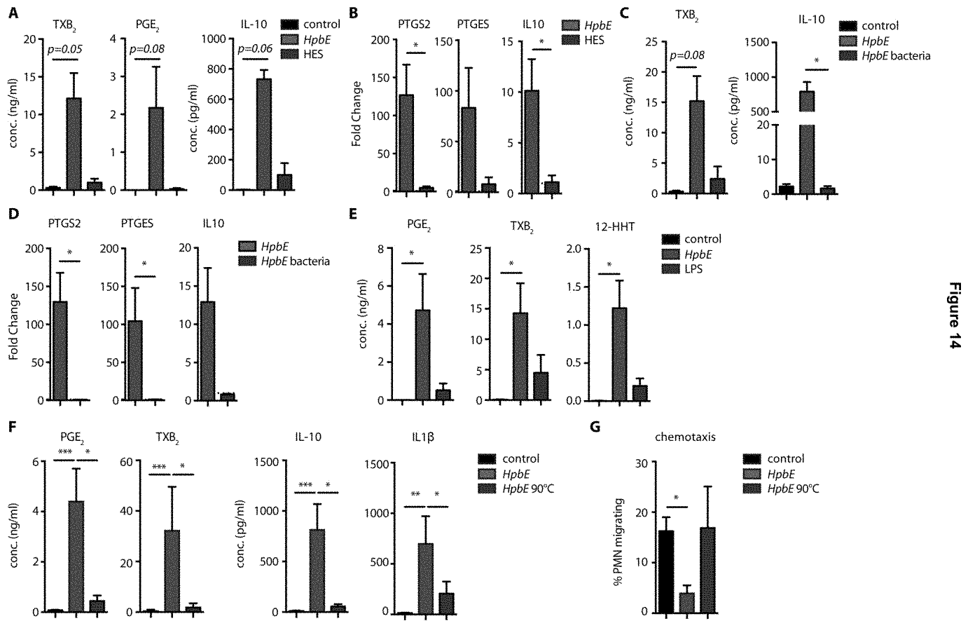

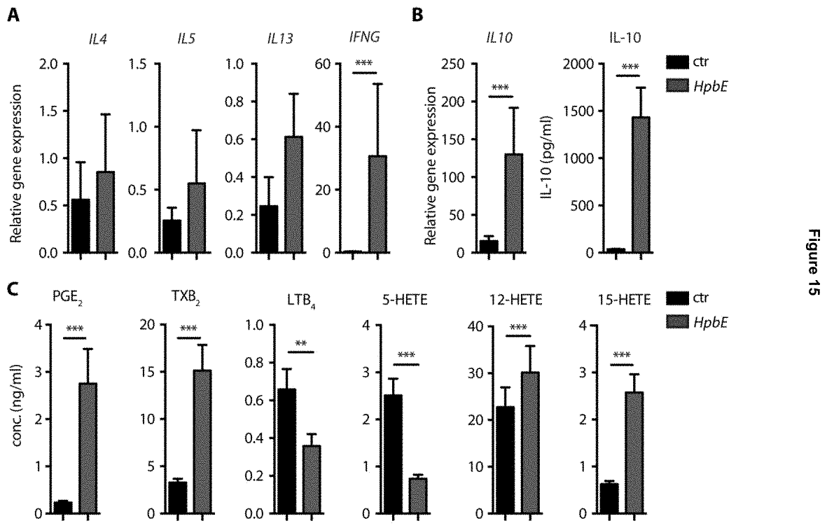

As larval stages of S. mansoni (S.m.) as well as excretory secretory products of Hpb adult stages (HES) can induce type 2-suppressive mediators, we compared S.m.- or HES-elicited effects on AA-metabolic pathways and IL-10 to those of HpbE. In contrast to the absence of 5-LOX protein during Hpb infection (FIG. 6C), 5-LOX was abundant in tissues of S.m.-infected mice (FIGS. 13A and B). Furthermore, an extract of S.m. larvae (SmE) failed to induce a shift from 5-LOX to COX metabolism and was less potent in triggering IL-10 production as compared to HpbE (FIGS. 13C and D). Similarly, adult-stage HES failed to induce the COX pathway as well as IL-10 (FIGS. 14A and B).

As changes in the microbiota contribute to the suppression of type 2 inflammation by Hpb infection, the inventors identified HpbE-associated bacteria and assessed whether these would exert similar effects as HpbE. However, COX metabolites, IL-10 and COX-pathway genes remained unaffected by treatment with HpbE-associated bacteria (FIGS. 14C and D). To further exclude that the HpbE-triggered induction of regulatory mediators was mainly due to LPS contamination, the inventors additionally quantified mediator profiles of macrophages treated with LPS at the concentration present in HpbE (60 ng/ml). However, LPS alone failed to significantly induce COX metabolites (FIG. 14E). Furthermore, heat treatment of HpbE abrogated the induction of COX metabolites and type 2-suppressive cytokines (FIG. 14F). Together this suggested that heat-labile components of HpbE larvae have a unique potential to induce type 2-suppressive COX metabolites in macrophages.

### Example 3.7: Hpb Larval Extract Remodels the AA Metabolism of Human Granulocytes

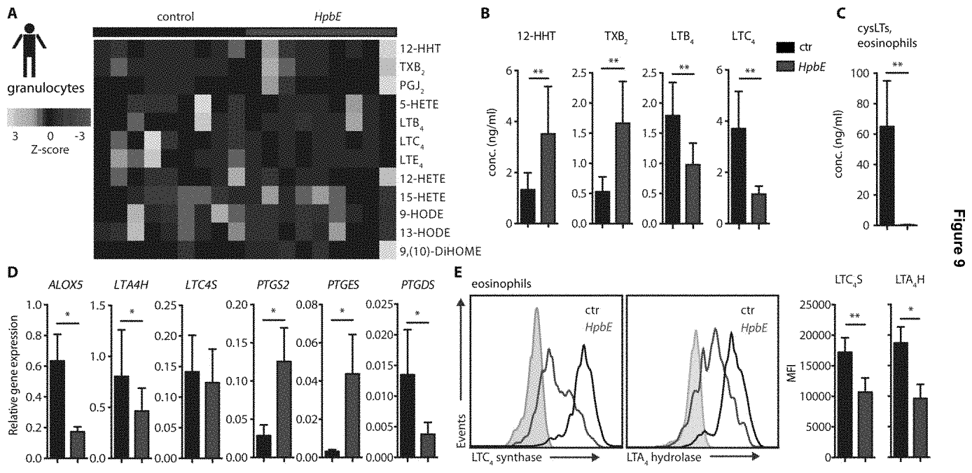

Together with macrophages, granulocytes represent a major source of pro-inflammatory eicosanoids during type 2 inflammation. Thus, the inventors used LC-MS/MS analysis to determine whether HpbE would affect the AA metabolism of human granulocytes. In line with the profiles observed for macrophages, granulocytes showed an induction of COX metabolites (particularly 12-HHT and TXB2) after treatment with HpbE (FIGS. 9A and 9B). Furthermore, the levels of 5-LOX metabolites (particularly cysLTs) were reduced by HpbE treatment in both mixed human granulocytes as well as in purified eosinophils (FIGS. 9B and 9C). Similar to HpbE-driven changes in AA metabolism genes in macrophages, the inventors observed a down-regulation of enzymes involved in the synthesis of pro-inflammatory mediators (ALOX5, LTA4H and PTGDS), whilst PTGS2 and PTGES were induced in HpbE-treated human granulocytes (FIGS. 9D and E).

### Example 3.8: HpbE does not Affect Type 2 Cytokines, but Modulates IFN-γ, IL-10 and Eicosanoids in PBMCs

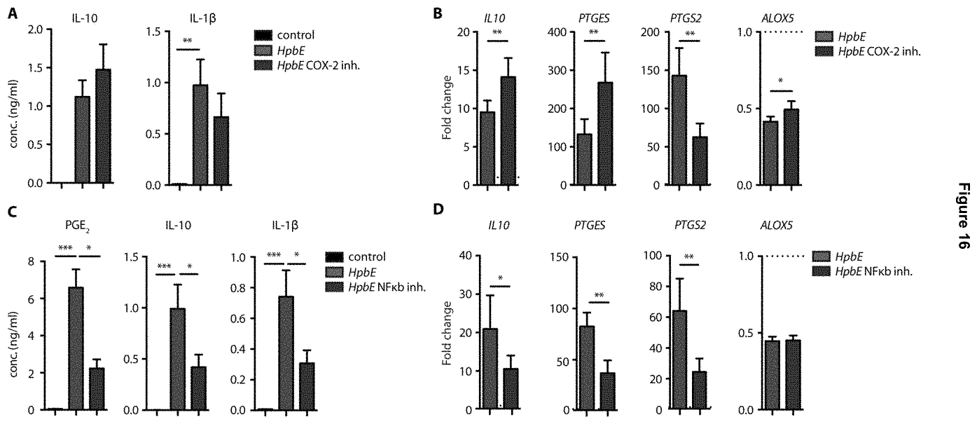

To test whether the regulatory potential of HpbE extended to type 2 cytokines, the inventors analyzed IL-4, IL-5 and IL-13 expression in human peripheral blood mononuclear cells (PBMCs) after treatment with HpbE. Type 2 cytokines were hardly affected by HpbE, which instead triggered a marked induction of IFN-γ and IL-10 (FIGS. 15A and B). In line with eicosanoid modulation in macrophages and granulocytes, HpbE treatment of PBMCs also triggered the synthesis of prostanoids (PGE2 and TXB2), whilst decreasing 5-LOX metabolites (5-HETE, and LTB4) (FIG. 15C). However, in contrast to macrophages and granulocytes, HpbE-treated PBMCs produced high levels of 12-/15-LOX metabolites (FIG. 15C), reminiscent of the AA metabolism during Hpb infection in vivo (FIGS. 1A and B). Thus, in both human and murine leukocytes as well as during infection in vivo, products of Hpb larvae induce an AA-metabolic profile, which is dominated by regulatory COX metabolites (e.g. PGE2) but lacks pro-inflammatory LTs (Table 1).

TABLE 1
Effects of HpbE on the AA metabolism in vivo and in myeloid cells
in vitro. Summary of LC-MS/MS and gene expression data for Hpb
infection in mice (intestinal culture supernatant or peritoneal
lavage) or treatment with Hpb larval extract (HpbE) of murine
or human leukocytes in vitro or during house dust mite allergy
in mice in vivo (bronchoalveolar lavage fluid).

| Setting: Hpb infection | COX | 5-LOX | 12/15-LOX |
|---|---|---|---|
| or HpbE treatment | pathway | pathway | pathway |
| Hpb infection in vivo | ++ | (+)/n.d. | ++ |
| Mouse BMDM in vitro | ++ | − | (−)/n.d. |
| Human MDM in vitro | ++ | − | − |
| Human PMN in vitro | ++ | −− | = |
| Human PBMCs in vitro | ++ | −− | ++ |
| HDM allergy in vivo | +/(n.d.) | − | − |

### Example 3.9: Activation of HIF1α by HpbE Mediates the Induction of a Type 2-Suppressive Mediator Profile

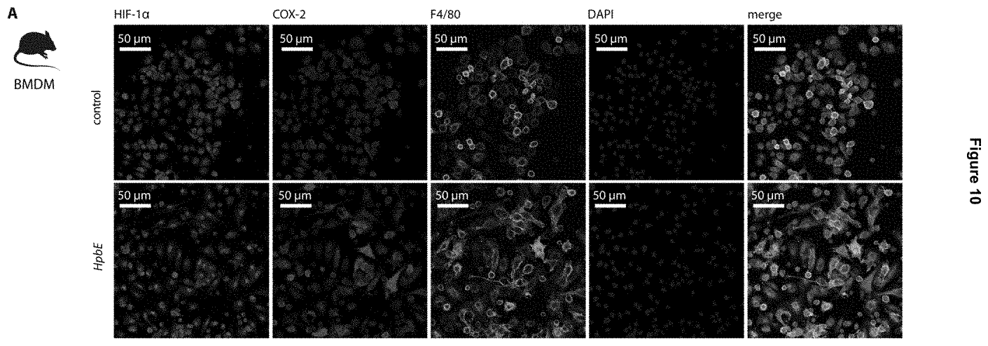

To identify mechanisms by which HpbE could trigger the production of type 2-suppressive mediators, the inventors targeted regulatory pathways genetically or pharmacologically and studied eicosanoid profiles and macrophage polarization. As our in vivo data suggested an involvement of HIF-1α in the Hpb-driven induction of COX-2, the inventors first assessed the effect of HpbE on HIF-1α activation and COX-2 expression. After treatment with HpbE, BMDM showed increased nuclear translocation of HIF-1α, increased expression of COX-2 and cellular redistribution of F4/80, indicative of an activated state (FIG. 10A). In contrast to wildtype BMDM, HIF-1α deficient BMDM (HIF-lafl/flxLysMCre) failed to upregulate prostanoids (TXB2 and PGE2) in response to HpbE, while the suppression of pro-inflammatory eicosanoids (PGD2 and LTB4) remained intact (FIG. 10B). In addition, HIF-1α deficient BMDM showed a reduced HpbE-driven induction of IL-6, TNFα and IL-10 as well as of the M2 markers Tgm2 and Arg1 (FIGS. 10C and D). Levels of Mrc1 were generally higher in BMDM lacking HIF-la, but HpbE down-regulated Mrc1 expression regardless of HIF-1α (FIG. 10D). Thus, the induction of type 2-suppressive mediators in BMDM was largely dependent on HIF-1α.

### Example 3.10: The HpbE-Driven Induction of Type 2-Suppressive Mediators Depends on p38 MAPK, COX and NFκβ

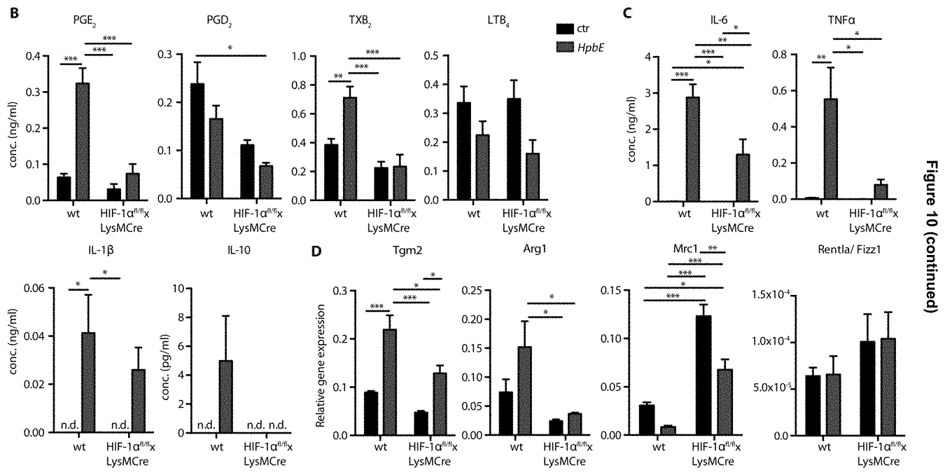

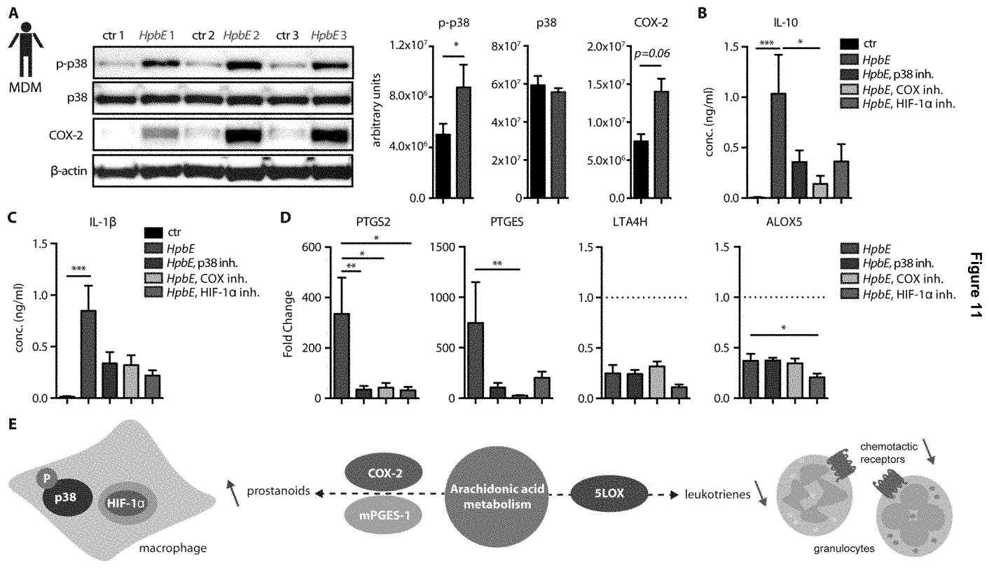

As HIF-1α is positively regulated by the p38 MAPK, the inventors studied the involvement of p38 signaling in the induction of type 2-suppressive mediators by HpbE. In human MDM, p38 was phosphorylated upon treatment with HpbE, correlating with the induction of COX-2 (FIG. 11A) and a p38 inhibitor (VX-702) abrogated the induction of IL-10, IL-1R and PGE2-synthetic enzymes (PTGS2 and PTGES) (FIG. 11B to D). In line with HIF-1α dependent regulation in murine BMDM, a pharmacological inhibitor of HIF-1α (acriflavine) attenuated the HpbE-induced expression of IL-10, IL-1β and COX pathway enzymes in human MDM (FIG. 11B to D). However, p38 and HIF-1α were not responsible for the modulation of the 5-LOX pathway (FIG. 11D).

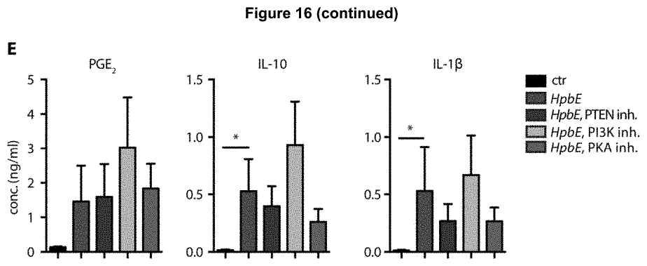

To investigate whether the HpbE-triggered production of IL-10 and IL-1β occurred downstream of the COX pathway, the inventors studied whether COX inhibitors could modify the induction of these cytokines. A non-selective COX inhibitor (indomethacin), but not a selective COX-2 inhibitor (CAY10404) reduced the induction of IL-10, IL-1β and PTGES (FIG. 11B to D, FIGS. 16A and B). In contrast, HpbE-triggered COX-2 expression was reduced by indomethacin as well as by selective inhibition of COX-2, while the suppression of the 5-LOX pathway remained largely unaffected (FIG. 11D and FIG. 16B).

As the transcription factor NFκβ and the kinases PI3 kinase, protein kinase A and PTEN can regulate AA-metabolic pathways, the inventors additionally assessed the contribution of these mechanisms to the induction of type 2-suppressive mediators by HpbE. Inhibition of NFκβ (by BAY 11-7085) significantly reduced PGE2, IL-10 and IL-1β production as well as gene expression of PGE2-synthetic enzymes and IL-10 in HpbE-treated human MDM (FIGS. 16C and D). In contrast, inhibitors of PI3 kinase, protein kinase A or PTEN did not interfere with the induction of PGE2, IL-10 or IL-1β (FIG. 16E).

### Example 3.11: TLR2, Dectin-1 and Dectin-2 Contribute to the Induction of the COX Pathway by HpbE

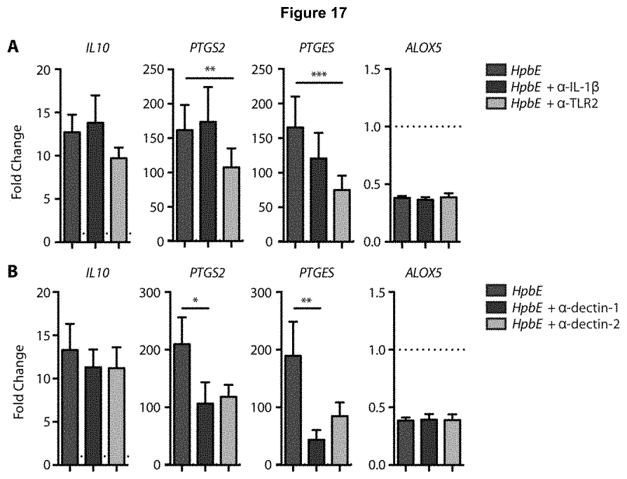

To further elucidate the upstream mechanisms underlying prostanoid- and cytokine modulation by HpbE, the inventors blocked IL-1β or pattern recognition receptors (PRRs; TLR2, dectins-1/2), which had all previously been linked to helminth-driven immuneregulation. Blockade of IL-1β neither affected the HpbE-driven modulation of IL-10 nor of AA-metabolic pathways (FIG. 17A). However, neutralizing antibodies against TLR2, dectin-1 or dectin-2 attenuated the induction of PGE2-synthetic enzymes by HpbE, whilst the modulation of IL-10 or 5-LOX was not affected (FIGS. 17A and B).

This suggested that HpbE induces the activation of p38 MAPK and transcription factors HIF-1α and NFκb, by engaging several PRRs, which together results in the induction of the COX pathway and increased production of type 2-suppressive mediators (FIG. 11E).

### Example 3.12: HpbE Inhibits Granulocyte Chemotaxis in Human Settings of Type 2 Inflammation

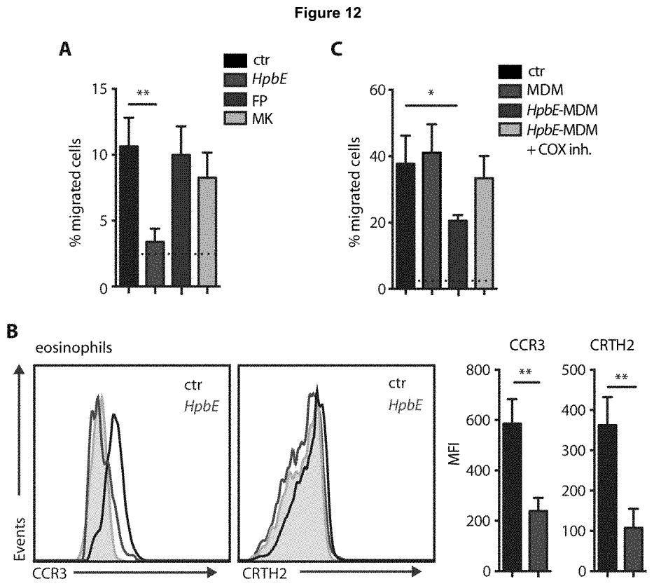

Eicosanoid-driven granulocyte recruitment represents a key event in type 2 inflammation. Thus, the inventors studied how HpbE would affect granulocyte recruitment in a clinically relevant setting of type 2 inflammation, in which AA metabolites play a major role. The inventors collected granulocytes and nasal polyp secretions from patients suffering from Aspirin exacerbated respiratory disease (AERD) and assessed the effects of HpbE on the migration of patient granulocytes towards nasal polyp secretions ex vivo. Pre-treatment of AERD granulocytes with HpbE resulted in a marked reduction in cell recruitment, an effect not achieved by anti-inflammatory drugs, which are used in the treatment of AERD (fluticasone propionate (FP), montelukast (MK)) (FIG. 12A). In keeping with the suppression of granulocyte chemotaxis, HpbE reduced surface levels of chemotactic receptors (C—C chemokine receptor type 3 (CCR3) and PGD2 receptor 2 (CRTH2)) on human eosinophils (FIG. 12B).

As for the heat-labile induction of type 2-suppressive mediators in macrophages, the suppression of granulocyte chemotaxis was lost upon heat treatment of HpbE (FIG. 14G).

To investigate whether COX metabolites released by HpbE-treated human macrophages could impact on granulocyte recruitment, we performed chemotaxis assays in the presence of conditioned media from MDM treated with HpbE and the non-selective COX-inhibitor indomethacin. In line with our in vivo data (FIGS. 7A and B), conditioned media from HpbE-treated human macrophages reduced granulocyte chemotaxis in a manner that was at least partially dependent on COX metabolites (FIG. 12C).

Thus, either directly or by acting on macrophages, HpbE can suppress the chemotaxis of granulocytes, including those from patients suffering from severe type 2 inflammation.

Materials and methods

### Mice

C57BLJ6J mice were bred and maintained under specific pathogen free conditions at the École Polytechnique Fédérale de Lausanne (EPFL) or at the Centre Hospitalier Universitaire Vaudois (CHUV). Alternatively, BALB/c and C57BL/6J mice were obtained from Charles River Laboratories (Sulzfeld, Germany). Unless stated otherwise, 6-12 weeks old mice of both sexes were used. All animal experiments were approved by the local authorities (Swiss Veterinary Office).

Heligmosomoides polygyrus Bakeri Infection and Preparation of Larval Extract

Infective stage-three larvae (L3) of Heligmosomoides polygyrus bakeri (Hpb) were obtained from the eggs of Hpb-infected mice as previously published (Camberis et al. (2003), Curr Protoc Immunol, Chapter 19, Unit 19.12). Mice were infected with 200 Hpb L3 larvae by oral gavage and small intestines were harvested 4-7 days post-infection for preparation of histological specimens or organ culture. For preparation of Hpb larval extract (HpbE), L3 larvae were homogenized in two cycles at 6.000 rpm for 60 seconds in a Precellys homogenizer using Precellys tough micro-organism lysing kits VK05 (Bertin Pharma). Remaining debris was removed by centrifugation (20 min, 14.000 rpm, 4° C.). When indicated, heat inactivated-HpbE (HpbE 90° C.) was prepared by heating at 90° C. overnight.

### House Dust Mite-Induced Allergic Airway Inflammation

Eight-weeks old female C57BL/6J mice were sensitized on day 0 by bilateral intranasal (i.n.) instillations of HDM extract from Dermatophagoides farinae (1 μg extract in 20 μl PBS; Stallergenes SA) and challenged on days 8-11 with 10 μg of the same extract dissolved in 20 μl PBS. Control animals received the same amount of PBS. HpbE treatment (5 μg Hpb extract in 20 μl PBS) was performed intranasally before sensitization and challenge. In the absence of HpbE treatment, the mice received 20 μl PBS. Three days after the last challenge, the airways of the mice were lavaged five times with 0.8 ml PBS. Aliquots of cell-free BAL fluid were frozen immediately with or without equal volumes of methanol. Viability, yield and differential cell count of BAL cells were performed as described before (Alessandrini et al. (2006), J. Allergy Clin. Immunol., 117:824-830).

### Human Blood and Tissue Samples

Peripheral blood mononuclear cells (PBMCs) or polymorphonuclear leukocytes (PMN) were isolated from the blood of healthy human donors or patients with Aspirin-exacerbated respiratory disease (AERD). Nasal polyp tissues were obtained during polypectomy of patients suffering from chronic rhinosinusitis with nasal polyps. Nasal polyp secretions were obtained from cultured nasal polyp tissues as described previously (Dietz et al. (2016), J. Allergy Clin Immunol, 139(4):1343-1354.e6). All blood and tissue donors participated in the study after informed written consent. Blood and tissue sampling and experiments including human blood cells were approved by the local ethics committee at the University clinic of the Technical University of Munich.

### Macrophage Cultures

Monocyte-derived macrophages (MDM) or bone marrow derived macrophages (BMDM) were generated by culture in the presence of human or murine recombinant GM-CSF (10 ng/ml) (Miltenyi Biotech) and human recombinant TGF-β1 (2 ng/ml) (Peprotech) as previously described (Esser-von Bieren et al. (2013), PLoS Pathog., 9:e1003771; Dietz et al. (2016), J. Allergy Clin Immunol, 139(4):1343-1354.e6). On day 6, cells were harvested and used for further experiments.

### Eicosanoid and Cytokine Analysis

Eicosanoids were quantified by liquid chromatography tandem mass spectrometry (LC-MS/MS) similar to a previously published method (Henkel et al. (2018); Allergy, doi:10.1111/a11.13700). Cytokines were quantified using commercially available multiplex assays or ELISA kits according to the manufacturer's instructions.

### Chemotaxis Assays

PMN were resuspended to a concentration of 1×10⁶ cells/ml in the presence of 100 ng/ml human GM-CSF (Miltenyi Biotech) and overnight stimulated with 10 μg/ml HpbE. When mentioned, PMN were pre-treated with 1 μM fluticasone propionate (Sigma-Aldrich), 10 μM montelukast (Cayman Chemical) or conditioned media from MDM stimulated overnight with 10 μg/ml Hpb extract+/−100 μM Indomethacin for 1 hour. PMN migration in response to nasal polyp secretions or a chemokine cocktail of 2 ng/ml RANTES, 20 ng/ml IL-8 (Miltenyi Biotech) and 2 ng/ml LTB4 (Cayman Chemical) was tested. Chemoattractants were placed in the lower wells of a chemotaxis plate (3 μm pore size; Corning). After mounting the transwell unit, 2×105 PMN were added to the top of each well and migration was allowed for 3 hours at 37° C., 5% CO2. The number of cells migrating to the lower well was counted microscopically. In some experiments, manual counting was validated by flow cytometry.

1. A cell-free larval preparation of Heligmosomoides polygyrus bakeri (Hpb) helminths, wherein said larval preparation is obtainable from cells of the L3-developmental stage larva of said Hpb helminths, wherein said larval preparation is capable of modulating the innate mammalian immune system.

2. The larval preparation according to claim 1, wherein said larval preparation is predominantly capable of modulating the innate mammalian immune system over the adaptive mammalian immune system.

3. The larval preparation according to claim 1, wherein said L3-developmental stage larva is an infective non-feeding larva, preferably said L3-developmental stage larva is between about 470-570 μm long.

4. The larval preparation according to claim 1, wherein said larval preparation comprises a polypeptide extract obtainable from cells of the L3-developmental stage larva of said Hpb helminths, preferably said polypeptide extract consisting essentially of polypeptides with molecular weight of 3 or more kDa; further preferably said polypeptide extract consisting essentially of polypeptides, wherein said polypeptides including oligomeric and/or monomeric polypeptides, with molecular weight of monomeric polypeptides in the range of about 3-70 kDa, most preferably said polypeptide extract consisting essentially of polypeptides, wherein said polypeptides including oligomeric and/or monomeric polypeptides, with molecular weight of monomeric polypeptides in the range of about 9-60 kDa.

5. The larval preparation according to claim 1, wherein said larval preparation comprises a solution of somatic proteins obtainable from cells of the L3-developmental stage larva of said Hpb helminths, preferably said somatic proteins consisting essentially of polypeptides with molecular weight of 3 or more kDa; further preferably said solution is aqueous, most preferably said somatic proteins consisting essentially of polypeptides, wherein said polypeptides including oligomeric and/or monomeric polypeptides, with molecular weight of monomeric polypeptides in the range of about 3-70 kDa, further most preferably said somatic proteins consisting essentially of polypeptides, wherein said polypeptides including oligomeric and/or monomeric polypeptides, with molecular weight of monomeric polypeptides in the range of about 9-60 kDa.

6. The larval preparation according to claim 1, wherein said larval preparation consists of an aqueous solution of a protein extract obtained from whole-larval homogenate of L3-developmental stage larva of Hpb helminths, preferably said polypeptide extract consisting essentially of polypeptides with molecular weight of 3 or more kDa; further preferably said protein extract consisting essentially of polypeptides including oligomeric and/or monomeric polypeptides with molecular weight of monomeric polypeptides in the range of about 3-70 kDa, most preferably said polypeptide extract consisting essentially of polypeptides including oligomeric and/or monomeric polypeptides with molecular weight of monomeric polypeptides in the range of about 9-60 kDa.

7. The larval preparation according to claim 1, wherein said larval preparation is capable of one or more of the following:
i) targeting phagocytic cells of said mammalian immune system; preferably said phagocytic cells: macrophages, neutrophils or dendritic cells (DC);
ii) modifying the activation of macrophages and/or granulocytes of mammalian immune system, preferably said granulocytes are eosinophils;
iii) acting intracellularly;
iv) modifying the activation of one or more of the leukotriene pathway of mammalian immune system;
v) decreasing the number of eosinophils and/or inhibiting the migration of granulocytes into tissue of said mammalian immune system;
vi) inhibiting tissue infiltration with neutrophils and/or eosinophils in mammals;
vii) binding to an iron atom associated with mammalian arachidonate 5-lipoxygenase (5-LOX) enzyme and/or iron atom associated with mammalian cyclooxygenase (COX) enzyme, preferably said mammalian enzyme is a human enzyme.
viii) reducing the expression and/or inhibiting one or more of the following:
(a) chemotactic receptors;
(b)cysteinyl leukotriene receptor 1 (CYSLTR1), preferably said CYSLTR1 is expressed by eosinophils;
(c) leukotriene C4 synthase (LTC4 synthase);
(d) arachidonate 5-lipoxygenase (5-LOX).

8. The larval preparation according to claim 1, wherein said larval preparation is capable of one or more of the following:
i) suppressing production of leukotrienes; preferably said leukotrienes are produced by myeloid cells including eosinophils;
ii) inducing of anti-inflammatory mediators, preferably said anti-inflammatory mediators comprise prostaglandin E2 (PGE2) and/or interleukin 10 (IL-10);
iii) reducing granulocyte recruitment and/or activation;
iv) inhibiting of arachidonate 5-lipoxygenase (5-LOX) having EC 1.13.11.34 enzymatic activity,
v) simultaneously capable of:
d) (i) and (ii); and/or
e) (i), (ii) and (iii); and/or
f) (i), (ii), (iii) and (iv).

9. The larval preparation according to claim 1, wherein said larval preparation comprises one or more of the following polypeptides:
i) a polypeptide, which is at least 60% or more identical to Hpb glutamate dehydrogenase polypeptide, said glutamate dehydrogenase polypeptide having UniProtKB Accession Number: A0A183FP08; preferably said polypeptide having EC 1.4.1.2 or EC 1.4.1.3 or EC 1.4.1.4 enzymatic activity; further preferably said polypeptide is capable of inducing of anti-inflammatory mediators according to claim 1;
ii) a polypeptide, which is at least 60% or more identical to Hpb ferritin polypeptide, said ferritin polypeptide having UniProtKB Accession Number A0A183FLG6 or A0A183FDM1, preferably said polypeptide having EC 1.16.3.1 enzymatic activity; further preferably said polypeptide is capable of suppressing production of leukotrienes according to claim 1;
iii) a polypeptide, which is at least 60% or more identical to Hpb aspartate aminotransferase polypeptide, said aspartate aminotransferase polypeptide having UniProtKB Accession Number: A0A183F107, preferably said polypeptide having EC 2.6.1.1 enzymatic activity;
iv) a polypeptide, which is at least 60% or more identical to Hpb tubulin alpha chain polypeptide; said tubulin alpha chain polypeptide having UniProtKB Accession Number: A0A183GTY4, A0A183F2N5, A0A183FGY7, A0A183FJ38 or A0A183G7U3;
v) a polypeptide, which is at least 60% or more identical to Hpb histone H2B polypeptide; said histone H2B polypeptide having UniProtKB Accession Number:
A0A183F3C5, A0A183FWH9, A0A183GMUO or A0A183GQR4;
vi) a polypeptide as in defined (i)-(v), wherein said polypeptide is orthologous or paralogous to the Hpb polypeptide as defined in (i)-(v);
vii) a polypeptide as in defined (i)-(v), wherein said polypeptide is a fragment of the Hpb polypeptide as defined in (i)-(v);
viii) combinations of (i)-(vii), preferably a combination of (i) and (ii).

10. The larval preparation according to claim 1, wherein said larval preparation comprises one or more of the following polypeptides, wherein said one or more polypeptides is selected from the group consisting of:
i) Hpb glutamate dehydrogenase polypeptide, said glutamate dehydrogenase polypeptide having UniProtKB Accession Number: A0A183FP08; preferably said polypeptide having EC 1.4.1.2 or EC 1.4.1.3 or EC 1.4.1.4 enzymatic activity; further preferably said polypeptide is capable of inducing of anti-inflammatory mediators according to claim 1;
ii) Hpb ferritin polypeptide, said ferritin polypeptide having UniProtKB Accession Number A0A183FLG6 or A0A183FDM1; preferably said polypeptide having EC 1.16.3.1 enzymatic activity; further preferably said polypeptide is capable of suppressing production of leukotrienes according to claim 1;
iii) Hpb aspartate aminotransferase polypeptide, said aspartate aminotransferase polypeptide having UniProtKB Accession Number: A0A183F107;
iv) Hpb tubulin alpha chain polypeptide, said tubulin alpha chain polypeptide having UniProtKB Accession Number: A0A183GTY4, A0A183F2N5, A0A183FGY7, A0A183FJ38 or A0A183G7U3;
v) Hpb histone H2B polypeptide, said histone H2B polypeptide having UniProtKB Accession Number: A0A183F3C5, A0A183FWH9, A0A183GMUO or A0A183GQR4;
vi) the polypeptide as in defined (i)-(v), wherein said polypeptide is a fragment of the Hpb polypeptide as in defined (i)-(v);
vii) combinations of (i)-(vi), preferably a combination of (i) and (ii).

11. The larval preparation according to claim 1, wherein said mammalian innate immune system is the human innate immune system.

12. A method of modulating the innate mammalian immune system in a subject in need thereof, comprising administering a polypeptide to said subject, wherein said polypeptide comprises a sequence at least 60% or more identical to a polypeptide obtainable from L3-developmental stage larva of Heligmosomoides polygyrus bakeri (Hpb) helminths selected from the group consisting of:
i) Hpb glutamate dehydrogenase polypeptide, said glutamate dehydrogenase polypeptide having UniProtKB Accession Number: A0A183FP08; preferably said polypeptide is capable of inducing of anti-inflammatory mediators according to claim 1
ii) Hpb ferritin polypeptide, said ferritin polypeptide having UniProtKB Accession Number A0A183FLG6 or A0A183FDM1; preferably said polypeptide is capable of suppressing production of leukotrienes according to claim 1;
iii) Hpb aspartate aminotransferase polypeptide, said aspartate aminotransferase polypeptide having UniProtKB Accession Number: A0A183F107;
iv) Hpb tubulin alpha chain polypeptide, said tubulin alpha chain polypeptide having UniProtKB Accession Number: A0A183GTY4, A0A183F2N5, A0A183FGY7, A0A183FJ38 or A0A183G7U3;
v) Hpb histone H2B polypeptide, said histone H2B polypeptide having UniProtKB Accession Number: A0A183F3C5, A0A183FWH9, A0A183GMUO or A0A183GQR4;
vi) the polypeptide as in defined (i)-(v), wherein said polypeptide is orthologous or paralogous to the Hpb polypeptide as defined in (i)-(v);
vii) the polypeptide as in defined (i)-(v), wherein said polypeptide is a fragment of the Hpb polypeptide as in defined (i)-(v).

13. A method for production of a cell-free L3-larval preparation of Heligmosomoides polygyrus bakeri (Hpb) helminths, said method comprising:
i) homogenizing L3-developmental stage larvae of Hpb helminths; preferably said homogenizing is a homogenizing of sedimented L3-developmental stage larvae of Hpb helminths;
ii) removing non-homogenized Hpb larval cells and cell debris followed by collecting resulting cell- and cell-debris free larval preparation, preferably said removing is carried out by centrifugation, wherein said larval preparation is collected in the form of supernatant;
iii) optionally, heat and/or acid treating said resulting larval preparation of (ii), preferably said heat treating is carried out at 60° C. or 90° C. for 24 hours and/or said acid treating is carried out with 1M HCl at 60° C. for 24 hours;
iv) isolating a polypeptide extract from said resulting larval preparation of (ii), preferably said polypeptide extract consisting essentially of polypeptides with molecular weight of 3 or more kDa; further preferably said polypeptide extract consisting essentially of polypeptides including oligomeric and/or monomeric polypeptides with molecular weight of monomeric polypeptides in the range of about 3-70 kDa, most preferably said polypeptide extract consisting essentially of polypeptides including oligomeric and/or monomeric polypeptides with molecular weight of monomeric polypeptides in the range of about 9-60 kDa; further most preferably said isolating is carried out by size exclusion chromatography, wherein said polypeptide extract is isolated in the form of protein fraction/s consisting essentially of polypeptides including oligomeric and/or monomeric polypeptides with molecular weight of monomeric polypeptides in the range of about 9-60 kDa;
v) optionally, testing said protein fractions of (iv) for a capacity to modulate the innate mammalian immune system and discarding protein fractions that are not able to modulate the innate mammalian immune system.

14. A cell-free L3-larval preparation of Heligmosomoides polygyrus bakeri (Hpb) helminths produced by the method of claim 13.

15. (canceled)

16. A method for eliciting or modulating an immune response in a subject in need thereof, comprising administering to said subject the larval preparation of Hpb helminths according to claim 1.

17. The method of claim 16, wherein said disease is selected from the group consisting of: chronic respiratory disease, steroid resistant airway inflammation, aspirin-exacerbated respiratory disease (AERD), nasal polyps, cystic fibrosis (CF), allergic rhino-conjunctivitis, atopic dermatitis, autoimmune and inflammatory disease, chronic inflammatory disease, rhinitis, diabetes; bronchitis, chronic bronchitis, mucopurulent chronic bronchitis, emphysema, MacLeod syndrome, panlobular emphysema, centrilobular emphysema, chronic obstructive pulmonary disease (COPD), chronic obstructive pulmonary disease with acute lower respiratory infection, chronic obstructive pulmonary disease with acute exacerbation, asthma, predominantly allergic asthma, atopic asthma, extrinsic allergic asthma, non-allergic asthma, idiosyncratic asthma, intrinsic nonallergic asthma, mixed asthma, asthmatic bronchitis, late-onset asthma, status asthmaticus, acute severe asthma, bronchiectasis, nasal polyps, cystic fibrosis (CF), allergic rhino-conjunctivitis, atopic dermatitis, autoimmune or inflammatory disease, steroid-resistant chronic respiratory disease or a steroid resistant airway inflammation, allergy.

18. A method for eliciting or modulating an immune response in a subject in need thereof, comprising administering to said subject the polypeptide according to claim 13.

19. The method of claim 18, wherein said disease is selected from the group consisting of: chronic respiratory disease, steroid resistant airway inflammation, aspirin-exacerbated respiratory disease (AERD), nasal polyps, cystic fibrosis (CF), allergic rhino-conjunctivitis, atopic dermatitis, autoimmune and inflammatory disease, chronic inflammatory disease, rhinitis, diabetes; bronchitis, chronic bronchitis, mucopurulent chronic bronchitis, emphysema, MacLeod syndrome, panlobular emphysema, centrilobular emphysema, chronic obstructive pulmonary disease (COPD), chronic obstructive pulmonary disease with acute lower respiratory infection, chronic obstructive pulmonary disease with acute exacerbation, asthma, predominantly allergic asthma, atopic asthma, extrinsic allergic asthma, non-allergic asthma, idiosyncratic asthma, intrinsic nonallergic asthma, mixed asthma, asthmatic bronchitis, late-onset asthma, status asthmaticus, acute severe asthma, bronchiectasis, nasal polyps, cystic fibrosis (CF), allergic rhino-conjunctivitis, atopic dermatitis, autoimmune or inflammatory disease, steroid-resistant chronic respiratory disease or a steroid resistant airway inflammation, allergy.
